(19) United States
(12) Patent Application Publication
(10) Pub. No.: US 2017/0023547 A1
(43) Pub. Date: Jan. 26, 2017

(54) CHEMICAL THREAT ASSESSMENT BY RAPID MOLECULAR PHENOTYPING

(71) Applicant: Vanderbilt University, Nashville, TN (US)

(72) Inventors: Jeremy L. Norris, Smyrna, TN (US); Richard M. Caprioli, Brentwood, TN (US)

(21) Appl. No.: 15/218,809

(22) Filed: Jul. 25, 2016

Related U.S. Application Data
(60) Provisional application No. 62/196,473, filed on Jul. 24, 2015.

(51) Int. Cl. G01N 33/50; G06F 19/26; G06F 19/18

(52) U.S. Cl. CPC G01N 33/502; G01N 33/5014; G06F 19/18; G06F 19/26; G01N 2570/00; G01N 2560/00

(57) ABSTRACT

Systems and methods for characterizing the cellular response of one or more animal cells to a chemical agent are described. The method includes the steps of: exposing one or more animal cells to a chemical agent; generating data representing an altered molecular phenotype of the one or more animal cells after exposure to the chemical agent using a multi-omic analysis; providing the data representing the altered molecular phenotype to a system comprising a processor; using the system to compare the data representing the altered molecular phenotype with data representing a normal molecular phenotype of the one or more animal cells; and using the system to output a characterization of the cellular response of the one or more animal cells to the chemical agent based on the results of comparing the data.

### CONTINUING APPLICATION DATA

This application claims the benefit of U.S. Provisional Application Ser. No. 62/196,473, filed Jul. 24, 2015, the disclosure of which is incorporated by reference herein.

### STATEMENT REGARDING FEDERALLY SPONSORED RESEARCH

This invention was made with government support under grant MCB-1411482 awarded by the National Science Foundation. The Government has certain rights in this invention.

### TECHNICAL FIELD

This disclosure relates generally to understanding how animal cells respond to perturbation by a chemical agent and more specifically to systems and methods employing a multi-omics platform to elucidate one or more mechanisms of action (MOA) of the chemical agent.

### BACKGROUND

There is a need to expand upon the way cellular responses to chemical perturbations are evaluated. The assessment of mechanisms of action (MOA) for exogenous compounds is currently time consuming, expensive, and generally accomplished through targeted analyses. This is exemplified by the identification of thalidomide's target of toxicity 50 years after observance of the teratogenic properties. Additionally, some compounds can elicit latent side effects, such as diethylstilbestrol. Even therapeutic compounds designed for specific targets induce pleiotropic effects, for example, statins. A comprehensive high-throughput strategy that both confirms hypothesized MOA and identifies potentially negative repercussions can significantly advance our molecular understanding of complex cellular responses. Such an approach requires exhaustive molecular profiling. Integration of transcriptomics, proteomics, and metabolomics facilitates a cohesive analysis of cellular response, and recent increases in analytical and computational capabilities make it feasible to obtain comprehensive data of a compound's MOA rapidly.

### SUMMARY

This disclosure relates generally to understanding how animal cells respond to perturbation by a chemical agent. An understanding of how cells respond to perturbation is essential for biological applications; however, most approaches for profiling cellular response are limited in scope to pre-established targets. More specifically, this disclosure relates to systems and methods employing a multi-omics platform to elucidate one or more mechanisms of action (MOA) of the chemical agent. The multi-omics platform can provide a global analysis of molecular MOA that will advance understanding of the complex networks constituting cellular perturbation and lead to advancements in areas, such as: infectious disease, developmental biology, pathophysiology, drug therapy, and toxicology. Indeed, using cisplatin as a test compound, the multi-omics platform was shown to be able to provide data to quantify over 10,000 unique, significant molecular changes in 30 days or less. These data provide excellent coverage of known cisplatin-induced molecular changes and previously unrecognized insights into cisplatin resistance and illustrates the utility of the multi-omics platform as a resource to understand complex cellular responses in a high throughput manner.

In one aspect, this disclosure relates to a method that can characterize a cellular response of one or more animal cells to a chemical agent. The method can include the steps of: exposing the one or more animal cells to the chemical agent; generating data representing an altered molecular phenotype of the one or more animal cells after exposure to the chemical agent using a multi-omic analysis; providing the data representing the altered molecular phenotype to a system comprising a processor; using the system to compare the data representing the altered molecular phenotype to data representing a normal molecular phenotype of the one or more animal cells; and using the system to output a characterization of a cellular response of the one or more animal cells to the chemical agent based on results of the comparing the data representing the altered molecular phenotype of the one or more animal cells to data representing the normal molecular phenotype of the one or more animal cells.

In another aspect, this disclosure relates to a system that can characterize a cellular response of one or more animal cells to a chemical agent. The system can include a non-transitory memory storing computer-executable instructions; and a processor to execute the computer-executable instructions. Upon execution of the computer-executable instructions, the system can receive data generated by a multi-omic analysis representing an altered molecular phenotype of one or more animal cells after exposure to a chemical agent; compare the data representing the altered molecular phenotype with data representing the normal molecular phenotype of the one or more animal cells; and output, to a display device, a characterization of the cellular response of the one or more animal cells to the chemical agent based on results of comparing the data.

### BRIEF DESCRIPTION OF THE FIGURES

FIG. 1 provides a block diagram of a system that can characterize a cellular response of one or more animal cells in a sample to a chemical agent, according to an aspect of the disclosure.

FIG. 2 provides a block diagram of operation of an example of the computing device of the system shown in FIG. 1.

FIG. 3 provides a process flow diagram of an example method for multi-omics analysis to facilitate the generation of data that can be performed by the testing apparatus of FIG. 1.

FIG. 4 provides a process flow diagram of an example method for characterizing the cellular response of one or more animal cells in a sample to a chemical agent, according to another aspect of the disclosure.

FIG. 5 provides a scheme showing a workflow that allows multi-omic analysis to be conducted.

FIG. 6 provides a scheme showing a proteomic analysis using the stable isotope labeling with amino acids in cell culture (SILAC) procedure.

FIG. 7 provides a scheme showing metabolomic analysis using liquid chromatography.

FIG. 8 provides a scheme showing transcriptomic analysis using RNAseq analysis.

FIG. 9 provides a graph showing the multi-omics platform for mechanism of action (MOA) construction. The 30-day procedure has three distinct phases: dose screen (days 0-3), discovery and validation (days 4-25), and mechanism construction (days 26-30). Phase 1 incorporates cell viability and molecular screens to establish protocols for the discovery phase. Phase 2 integrates proteomics, metabolomics, and transcriptomics to determine molecular changes correlated with compound exposure. In Phase 3, network analysis of all statistically significant changes drives construction of a comprehensive MOA.

FIGS. 10A-G provide graphs and images from phase 1: dose screening (days 0-3). (A) Two stages of the screening are illustrated. The phenotypic screens evaluate physiological changes to narrow the exposure to a small number of responsive conditions. The molecular screen focuses these possibilities to the optimal conditions. The green boxes represent theoretical dose ranges. (B) This cartoon conceptualizes the approach to determine optimal exposure conditions. The green box highlights the ideal target dose range, one that stimulates the greatest molecular response while maintaining a tolerable level of cell viability. (C and D) Phenotypic screening results for cisplatin at 24 h show C) relative caspase activation (ApoONE) and D) cell viability (CellTiter Glo). The green boxes demonstrate effective responses. (E) Qualitative evaluation of the cell viability at 24 h confirms an optimal dose range. Green: live cells; red: dead cells. (F) MALDI FTICR MS spectra from a 6 h exposure illustrate molecular differences in control (top, black) and 50 μM cisplatin-treated samples (bottom, green). The inset highlights differences in the molecular signatures of the control and treated samples within a selected m/z region (see asterisks). (G) This graph summarizes the results of the molecular screen. 753 The green box indicates the selected dose for Phase 2 discovery experiments.

FIGS. 11A-C provide diagrams showing phase 2: discovery results (Days 4-25). A) Significantly changed proteins (based on gene symbol) and B) identified (top) and significantly changed (bottom) metabolites show the overlap across the modalities and time. For visual simplicity, 3 out of 4 time points are shown. Abbreviations: LF—label-free; HILIC—hydrophilic liquid interaction chromatography; RP—reverse phase chromatography. (C) Transcriptomic data show the overlap of significantly changed transcripts across time. (D) A cross-platform comparison of unique, significantly changed species shows the overlap between transcriptomics and proteomics.

FIGS. 12A-D provide schematic representations of phase 3: mechanism construction (days 26-30). (A) The cisplatin canonical MOA generated from a literature survey (green). (B) A vignette of the intrinsic apoptosis pathway illustrates directional fold changes and detection status from the empirical data. Abbreviations: ERK: ERK1/2; ERKP: ERK1 pThr202/pTyr204 and ERK2 pThr185/pTyr187; p53P: p53 pSer392. (C) This workflow conceptualizes the reconstruction of networks from seeding species. (D) An overlay of the ECN (green) and the DDN (blue) demonstrates the comprehensive nature of empirical mechanism construction beyond the canonical mechanism.

FIGS. 13A-F provide schemes and graphs showing steps beyond the primary MOA. (A) The CUL4B/HUWE1 pathway (pink) can modulate the intrinsic apoptosis pathway (green). (B) Relative caspase activation (top) and % viability (ATP levels; bottom) of 50 μM cisplatin-treated cells compared to untreated. (C) Superimposition of the ERN (red) and the DDN (blue) demonstrates capture of known and potentially novel resistance mechanisms. (D) ATP1A1 regulates Ncx1 activity (orange), which can affect the regulation of apoptosis. (E) This pathway illustrates the estrogen-induced cisplatin resistance mechanism (teal). (F) The STIP1 cascade (purple) initiates STIP-1 and PRNP interaction and endocytosis, which ultimately leads to phosphorylation of BAD and inhibition of apoptosis.

FIG. 14 provides a schematic representation of the integrated molecular response to cisplatin perturbation. The cisplatin canonical MOA (green) determined by multiple groups over a 20 year span integrates with the empirically elucidated pathways: CUL4B/HUWE1 pathway (pink) ATP1A1 pathway (orange); STIP1 cascade (purple); estrogen resistance pathway (teal). Graying represents pathways only from canonical. The comprehensive mechanism obtained in 30 days or less captures possible resistance.

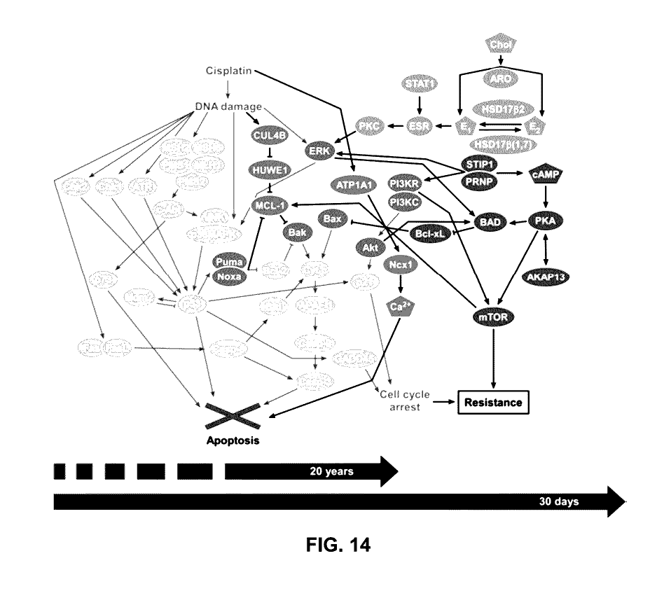

### DETAILED DESCRIPTION

In this disclosure, systems and methods are provided to characterize the cellular response of one or more animal cells to a chemical agent. The systems and methods described herein provide the capability to identify unique molecular phenotypes caused by exposure to the chemical agent using high-throughput multi-omic analysis (e.g., a laser-based mass spectrometry analysis, transcriptomic analysis, etc.). Cellular events, such as effects of binding, catalysis, and post-translational alterations, can be measured as perturbations in the molecular phenotypes characterized according to the specific cellular perturbations that make up that phenotype. A database can provide the basis for subsequent rapid assessment of challenge compounds and identification of MOA, and provides the necessary throughput to assess the MOA of a chemical agent in 30 days or less.

### DEFINITIONS

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such. Furthermore, the recitation of numerical ranges by endpoints includes all of the numbers subsumed within that range (e.g., 1 to 5 includes 1, 1.5, 2, 2.75, 3, 3.80, 4, 5, etc.).

Throughout this application, the term “about” is used to indicate that a value includes the standard deviation of error for the device or method being employed to determine the value, except that the value will never deviate by more than 5% from the value cited.

The term “biomolecule(s)” as used herein refers to a protein, a peptide, a nucleic acid, a sugar, or a lipid that exists extracellularly or intracellularly. The biomolecules include any organic molecules synthesized by the animal cells used herein. The term “nucleic acid” included in the biomolecule(s) as used herein refers to a single-stranded or double-stranded nucleic acid(s) containing at least 10, preferably 50, 300, 500, or 1000 or more nucleotides, and preferably interacts with a specific low-molecular-weight compound. A nucleic acid may be DNA or RNA. Examples of RNA include tRNA, ribosome RNA, and ribozyme. Examples of a sugar included in the biomolecule(s) as used herein include polysaccharides that preferably interact with specific low-molecular-weight compounds. Examples of such sugars include proteoglycans or derivatives thereof such as hyaluronic acid, chitin, heparan sulfate, keratan sulfate, dermatan sulfate, sialic acid, and chondroitin sulfate.

As used herein, the term “polypeptide” refers to an oligopeptide, peptide, or protein, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, all “mimetic” and “peptidomimetic” polypeptide forms, and retro-inversion peptides (also referred to as all-D-retro or retro-enantio peptides). Generally, a peptide has less than 30 amino acids, whereas a protein has more than 30 amino acids, though this is an approximate dividing line between the two.

As used herein, the term “lipid” refers to naturally occurring fats, waxes, sterols, monoglycerides, diglycerides, triglycerides, and phospholipids. Examples of the term “lipid” included in the biomolecule(s) as used herein include lipids that are contained in the above illustrated organisms, and preferably interact with specific low-molecular-weight compounds. Examples of such lipid include phospholipids such as a sphingophospholipid and a glycerophospholipid, glycolipids such as a sphingoglycolipid and a glyceroglycolipid, and conjugated lipids that form extracellular or cell membranes, such as a lipoprotein lipid, a sulpholipid and a galactolipid.

As used herein, the term “memory” refers to any type of non-transitory (e.g., enduring or long-lived) computer-readable media. Examples of such non-transitory computer-readable media include register memory, processor cache, random access memory (RAM), read only memory (ROM). In other words, all types of computer-readable media are included except for propagating transitory signals, which are excluded by this definition.

### Chemical Agents

Aspects of the present disclosure can be used to characterize the cellular response to a wide variety of different chemical agents. Examples of chemical agents include can include minerals, biomolecules, and small organic molecules. Chemical agents can also include pharmaceutical agents, toxins, and chemical warfare agents. The chemical agent can be either known or unknown a priori. The chemical agent can be either known or unknown a priori.

In some examples, the chemical agent is a pharmaceutical agent. Examples of pharmaceutical agents include antineoplastic agents, such as cisplatin, paclitaxel, sagopilone, docetaxel, rapamycin, doxorubicin, daunorubicin, idarubicin, epirubicin, capecitabine, mitomycin c, amsacrine, busulfan, tretinoin, etoposide, chlorambucil, chlormethine, melphalan, and benzylphenylurea compounds; steroidal compounds, such as natural and synthetic steroids, and steroid derivatives, such as cyclopamine; antiviral agents, such as aciclovir, indinavir, lamivudine, stavudine, nevirapine, ritonavir, ganciclovir, saquinavir, lopinavir, and nelfinavir; antifungal agents, such as itraconazole, ketoconazole, miconazole, oxiconazole, sertaconazole, amphotericin b, and griseofulvin; antibacterial agents, such as quinolones, e.g., ciprofloxacin, ofloxacin, moxifloxacin, methoxyfloxacin, pefloxacin, norfloxacin, sparfloxacin, temafloxacin, levofloxacin, lomefloxacin, and cinoxacin; antibacterial agents, such as penicillins, e.g., cloxacillin, benzylpenicillin, and phenylmethoxypenicillin; antibacterial agents, such as aminoglycosides, e.g., erythromycin and other macrolides; antitubercular agents, such as rifampicin and rifapentine; and anti-inflammatory agents such as ibuprofen, indomethacin, ketoprofen, naproxen, oxaprozin, piroxicam, and sulindac.

In some examples, the chemical agent is a toxic agent (i.e., toxin or toxicant). Toxins are poisonous substances produced within living cells or organisms, whereas toxicants are synthetic compounds created by artificial processes. Examples of toxins include bacterial toxins (both endotoxins and exotoxins), venoms, hemotoxins, and phototoxins. Examples of toxins include lipopolysaccharide (LPS), Clostridium difficile toxins TcdA and TcdB, H. pylori VacA toxin, ricin, and anthrax lethal toxin. In some examples, the animal cells are exposed to the toxin as a result of the presence of a microorganism such as a bacteria or virus present in the cell culture.

In some examples, the chemical agent is a chemical warfare agent. Examples of chemical warfare agents include blister agents such as sulfur mustard, nitrogen mustard, lewisite, and phosgene oxime; blood agents such as hydrogen cyanide, cyanogen chloride and arsine; nerve agents, including organophosphorus compounds such as tabun (O-ethyl dimethylamidophosphorylcyanide), sarin (isopropyl methylphosphonofluoridate), soman (pinacolyl methylphosphonofluoridate), GF (cyclohexyl methylphosphonofluoridate), and VX (O-ethyl S-diisopropylaminomethyl methylphosphonothiolate), riot control agents such as pepper spray and tear gas (e.g., ortho-chlorobenzylidene-malononitrile), psychotomimetic agents such as BZ (3-quinuclidinylbenzilate), phencyclidine, and lysergic acid diethylamide (LSD), and toxins.

In some examples, one or more animal cells are exposed to a single, homogenously applied dose. In other embodiments, a plurality of animal cells are exposed to different concentrations of the chemical agent, or one or more animal cells can be evaluated at a plurality of different times to determine the effect(s) of the chemical agent on cellular responses over time. When exposed to different concentrations, a gradient of the field of the chemical agent can be provided. The ability to obtain a molecular image of a well containing a field of cells using imaging mass spectrometry makes possible experiments that involve pinpoint central administration of nL to pt volumes of the chemical agent to viable animal cells in a low-volume medium for a specific period of time to allow diffusion and mechanical distribution, resulting in an exposure gradient. Cell metabolism could then be stopped by, for example, freezing the wells followed by molecular mapping of the cellular compounds using multi-omic analytic methods. An advantage of this approach is that important information on the effect of the dose, exposure time, and possible cell-cell communication can be assessed in a single experiment.

### System for Characterizing the Cellular Response of One or More Animal Cells

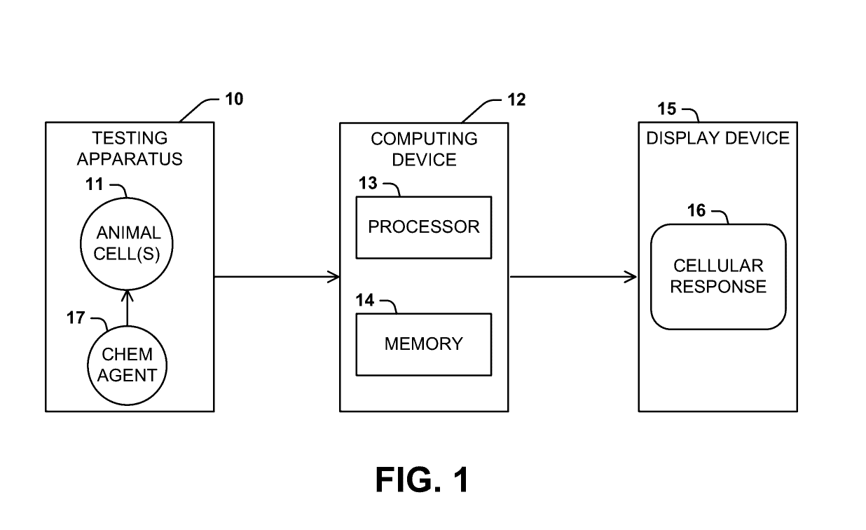

An example of a system that is directed to characterizing the cellular response of one or more animal cells to a chemical agent is shown in FIG. 1. The cellular response can be related to molecular phenotypes expressed in cells within a sample. As shown in FIG. 1, the system includes a testing apparatus 10, a computing device 12, and a display device 15.

The testing apparatus 10 can be used to expose a sample (e.g., including one or more animal cells 11) to the chemical agent 17. For example, the one or more animal cells 11 can comprise a portion of animal tissue. The chemical agent 17 can be a known chemical agent or an unknown chemical agent. The testing apparatus 10 can apply a homogenous dose of the chemical agent 17 to the sample or a gradient dose of the chemical agent 17 to the sample (e.g., where different portions of the one or more animal cells 11 are exposed to different concentrations of the chemical agent 17). After the sample is exposed to the chemical agent 17, data related to the cellular response can be collected. The data can be collected, for example, in response to a multi-omics analysis based on the exposure. The multi-omics analysis can include at least one of proteomic analysis, metabolomic analysis, and transcriptomic analysis. Multi-omic analysis is analysis using one or more of these methods, or in some embodiments, at least two or more of these methods. One type of such analysis can be conducted using desorption ionization mass spectrometry analysis, such as matrix-assisted laser desorption/ionization (MALDI) mass spectrometry. A MALDI mass spectrometer can be included in the testing apparatus 10. The MALDI mass spectrometer can be, for example, a high dynamic range (HDR) MALDI. The testing apparatus 10, in some examples, can include a well plate having a plurality of wells with different portions of the one or more animal cells 11 cultured therein and/or a high throughput apparatus that includes a robot arm for analysis of the plurality of wells in the well plate. The data can represent an altered molecular phenotype of the one or more animal cells 11 after exposure to the chemical agent 17. This altered data can be input into a computing device 12, which can characterize the cellular response of at least a portion of the sample to the chemical agent 17. In some instances, the animal cells 11 can be screened to select optimal treatment conditions before the data representing the altered molecular phenotype is generated.

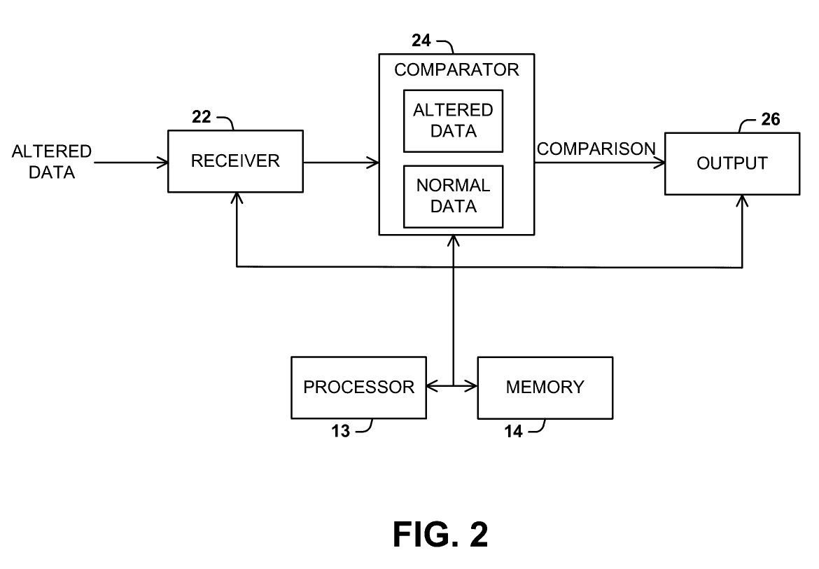

The computing device 12 includes at least a memory 14 and a processor 13. The memory 14 stores computer-executable instructions and the processor 13 executes the computer-executable instructions to accomplish the characterization of the cellular response, shown in greater detail in FIG. 2. Upon execution of the instructions, the processor 13 can generate at least a receiver 22, a comparator 24, and an output 26. The receiver 22 can receive data generated by a multi-omics analysis representing the altered molecular phenotype of one or more animal cells 11 after exposure to a chemical agent 17 (or “altered data”). In some instances, the receiver 22 can perform any required preprocessing of the altered data. The receiver 22 can feed the altered data to the comparator 24, which can retrieve data representing the normal molecular phenotype of the one or more animals (or “normal data”). For example, the comparator 24 can retrieve the normal data from the memory 14 or other data store associated with the computing device (e.g., the normal data can be stored in a database). However, the comparator 24 can retrieve the normal data from other sources, such as a remote data store or data repository. As an example, the data representing the normal molecular phenotype of the one or more animal cells can be obtained concurrently with the data representing the altered molecular phenotype using corresponding isotopically animal cells.

The comparator 24 can determine the cellular response by comparing the altered data after exposure to the chemical agent to the normal data and identifying differences therebetween. Results of the comparison by the comparator 24 can be provided as an output 26 to the display device 14. The results can be formatted in a manner that is easily perceivable by a human (e.g., a visualization, a sound, etc.). The display device 14 can provide a visualization of the characterization (e.g., an image, a chart, a graph, etc.). As an example, the display device 14 can be a computer monitor or other visual output. However, the output 26 is not confined to a visible image so that the display device 14 can be any type of device that can convey the output 26 to a human user. For example, the display device 14 can include one or more speakers or other audio conveying modality.

### Method for Characterizing the Cellular Response of One or More Animal Cells

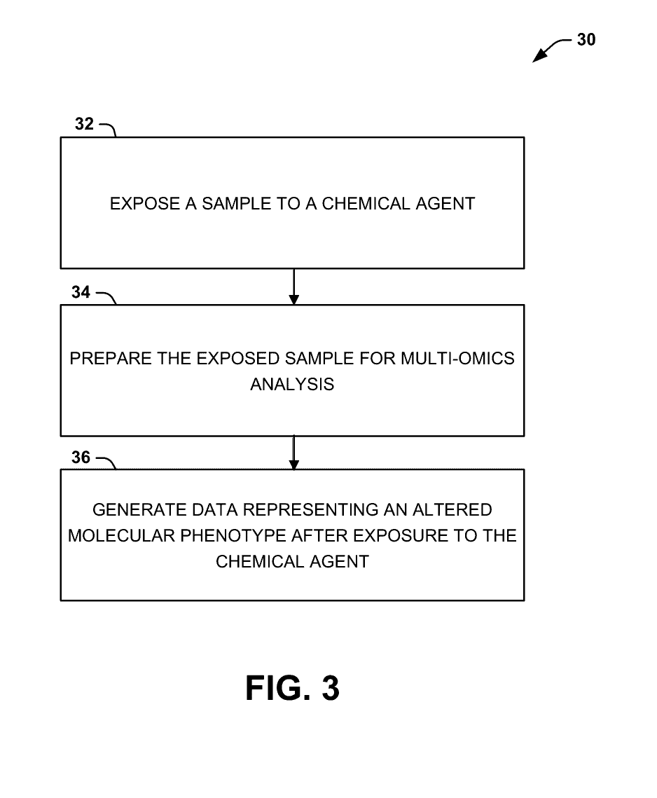

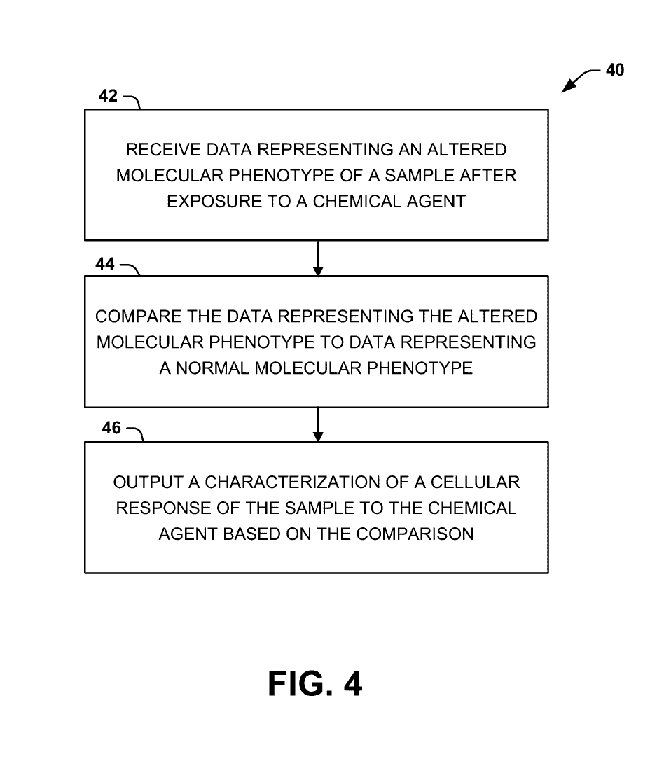

Example methods 30 and 40 that can be executed by the system illustrated in FIGS. 1-2 to characterize the cellular response of one or more animal cells to a chemical agent are shown in FIGS. 3 and 4, respectively. The cellular response can be related to molecular phenotypes expressed in cells within a sample. The methods 30 and 40 are illustrated as process flow diagrams with flowchart illustrations. For purposes of simplicity, the methods 30 and 40 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 30 and 40.

FIG. 3 illustrates an example method 30 for multi-omics analysis to facilitate the generation of data that can be performed by the testing apparatus 10 of FIG. 1. At 32, the sample can be exposed to a chemical agent (e.g., as a single dose or a gradient dose). At 34, the exposed sample can be prepared for multi-omics analysis. At 36, data representing an altered molecular phenotype after exposure to the chemical agent can be generated.

FIG. 4 illustrates an example method 40 for characterizing the cellular response of one or more animal cells in a sample to a chemical agent. The method 40 can be performed by the computing device 12 of FIG. 1. At 42, data representing the altered molecular phenotype can be received. At 44, the data representing the altered molecular phenotype can be compared to data representing a normal molecular phenotype. At 46, a characterization of the cellular response of the sample to the chemical agent based on the comparison can be output. The output can be displayed by the display device 15 of FIG. 1.

### Molecular Phenotypes and Multi-Omic Analysis

The molecular phenotypes represent the spectrum of different biomolecules determined to be present in the animal cell(s) 11, while the cellular response represents the changes in expression of those biomolecules after exposure to the chemical agent 17. The normal molecular phenotype represents the biomolecules present in a cell that has not been exposed to a chemical agent, while the altered molecular phenotype represents the biomolecules present in a cell that has been exposed to a chemical agent 17. Examples of biomolecules include polypeptides, amino acids, nucleic acids, sugars, lipids, vitamins, and various other cellular metabolites. The types of biomolecules identified as being part of the molecular phenotype vary depending on the methods being used to carry out the multi-omic analysis, and the methods, in turn, depend on the type of analysis being conducted.

In some instances, the normal molecular phenotype of the one or more animal cells 11 is limited to the molecular phenotype associated with a specific biological pathway. A wide variety of biological pathways are known to exist in cells, and consist of a variety of proteins and other factors which work together to achieve a particular biochemical result, such as energy generation or regulation of cell growth or apoptosis. Limiting analysis to the molecular phenotype associated with a specific biological pathway can be useful in at least a couple of ways. First, identification of portions of the altered molecular phenotype that correspond to known biological pathways can be excluded so that new biological pathways associated with biomolecules not associated with a known biological pathway can be identified. Second, in the case of an unknown chemical agent, identification of the biological pathways affected by the chemical agent can be used to help identify the chemical agent, or possibly identify routes through which undesirable effects of the chemical agent could be countered.

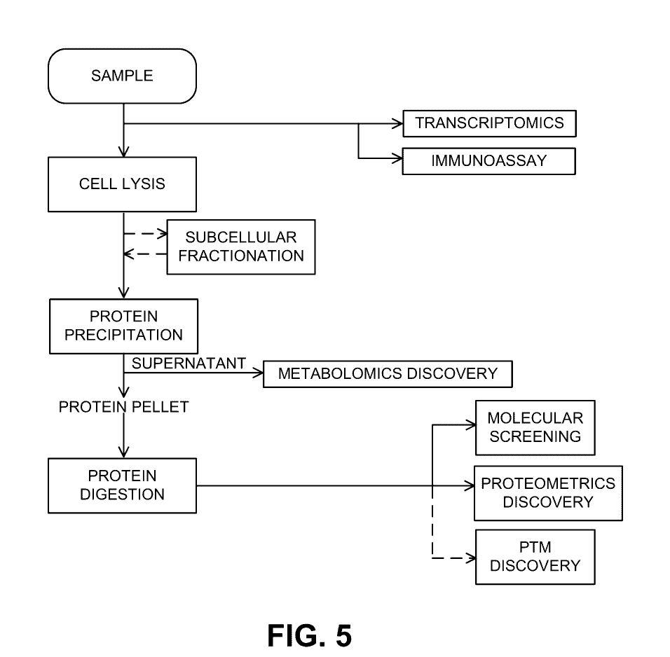

Multi-omic analysis is analysis using one or more of these methods, or in some instances, at least two or more of these methods. These analyses can be carried out separately, or they can be carried out using a unified sample preparation strategy as illustrated in FIG. 5. FIG. 5 outlines how a single sample of animal cells can be treated to allow transcriptomic, immunoassay, metabolomic, and proteomic analysis to be carried out from the same sample of animal cells.

FIG. 5 also makes reference to a molecular screening step. Molecular screening represents the step of screening the cells before carrying out the multi-omic analysis in order to select optimal treatment conditions before generating data representing an altered molecular phenotype of the one or more animal cells after exposure to the chemical agent. Screening can be carried out using morphological characterization of cells using microscopy, determination of cell viability markers such as caspase, and/or can be carried out using desorption ionization mass spectrometry. Screening is useful to determine if the dose of the chemical agent is sufficient to generate readily detectable changes without killing the animal cells so rapidly that changes in the phenotype, and in particular those relating to particular biochemical pathways, cannot be identified.

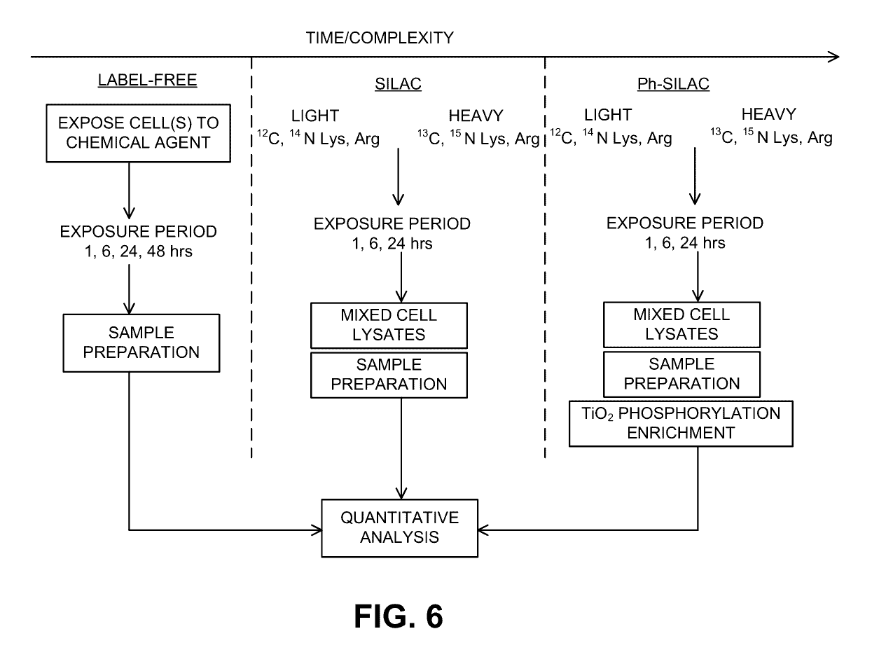

FIG. 6 provides a scheme illustrating a proteomic approach to analysis of animal cells exposed to a chemical agent. Proteomics is the large-scale study of proteins, and in this case is the study of a large number of proteins making up the molecular phenotype of animal cells. Proteins are the main components of the physiological metabolic pathways of cells, and as such their study after exposure of an animal cell to a chemical agent is particularly revealing of the mechanisms through which the chemical agent acts upon the biochemical pathways of the animal cell. The main methods used to study proteins are antibodies and mass spectrometry. There are several specific techniques and protocols that use antibodies for protein detection. The enzyme-linked immunosorbent assay (ELISA) has been used for decades to detect and quantitatively measure proteins in samples, and Western blot can be used for detection and quantification of individual proteins, where a complex protein mixture is separated using SDS-PAGE and then the protein of interest is identified using an antibody. However, mass spectrometry is more suited for high-throughput analysis of a large number of proteins.

One proteomics method particularly suitable for high-throughput analysis uses stable isotope tags to differentially label proteins from two different complex mixtures. Using this method, the proteins within a complex mixture are labeled first isotopically, and then digested to yield labeled peptides. The labeled mixtures are then combined, the peptides separated by multidimensional liquid chromatography and analyzed by tandem mass spectrometry. This method is illustrated in FIG. 6, which shows the use of stable isotope labeling with amino acids in cell culture (SILAC). Using this method, heavy and light versions of carbon and nitrogen are used in cell cultures to create animal cells whose proteins can be readily distinguished. This allows an internal control that allows cell cultures treated with a chemical agent, and normal cells not treated with a chemical agent, to be readily evaluated simultaneously. Accordingly, in some aspects, the data representing the normal molecular phenotype of the one or more animal cells is obtained concurrent with the data representing the altered molecular phenotype using corresponding radiolabeled animal cells.

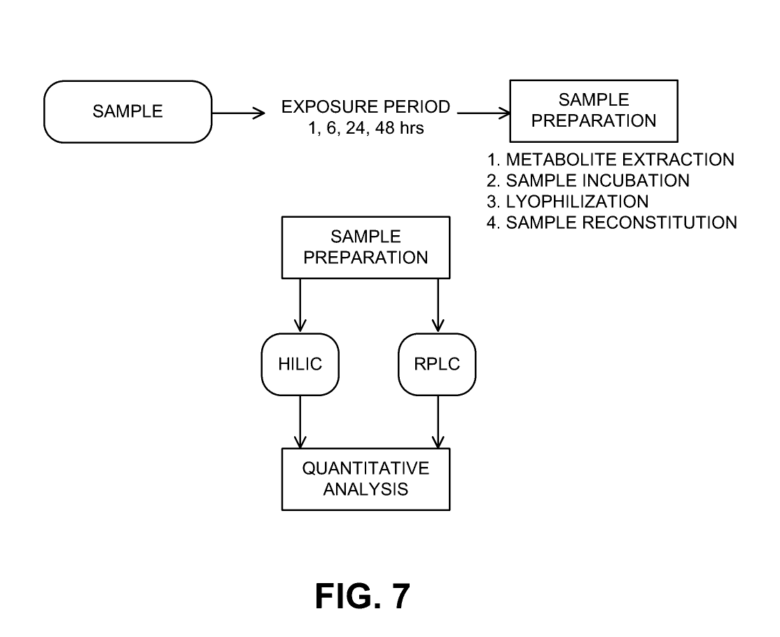

FIG. 7 provides a scheme showing a method for carrying out a metabolomic analysis. Metabolomic analysis is useful for looking at a wide range of biomolecules and in particular metabolites that are generally smaller than the proteins evaluated in a proteomic analysis. Metabolites are the intermediates and products of metabolism. Within the context of metabolomics, a metabolite is generally defined as any molecule less than 1 kDa in size. Examples of metabolites that can be evaluated using metabolomic analysis include amino acids and small peptides such as arginine, citrulline, tryptophan, phenylalanine, glutamyl-threonine, glutathione, D-pyroglutamic acid, and oxidize glutathione; lipids such as oleamide, palitamide, sphingosine, sphinganine, diacylglycerophosphocholine, lysophosphatidylcholine, linoleic acid, linolenelaidic acid, phosphocholine, and palmitoylsphingosine; vitamins and vitamin derivatives such as pyridoxine (B₆), panthothenic acid (B₅) and flavin adenine dinucleotide (FAD); nucleic acids such as 2′-deoxyguanosine, uridine 5′-diphosphate (UDP), 5′-S-methylthioadenosine, adenosine 5′-monophosphate (AMP), nicotinamide adenine dinucleotide (NAD⁺), 5-phospho-D-ribose 1-diphosphate, cyclic adenosine diphosphate ribose, and uridine diphosphate-N-acetylgalactosamine; sialic acids such as N-acetyl-D-lactosamine and N-acetyl-D-galactosamine; and other metabolites such as carnitine, acetylindole, coumaric acid, phenacylamine, acetyl-L-carnitine, 2-amino-1-phenylethanol, and S-adenosyl-L-methionine. Metabolomic analysis is typically conducted using a separation step using one or more forms of liquid chromatography (e.g., high performance liquid chromatography), such as hydrophilic interaction liquid chromatography and/or reverse phase liquid chromatography, followed by compound identification using mass spectrometry. However, metabolites can also be separated using gas chromatography or capillary electrophoresis.

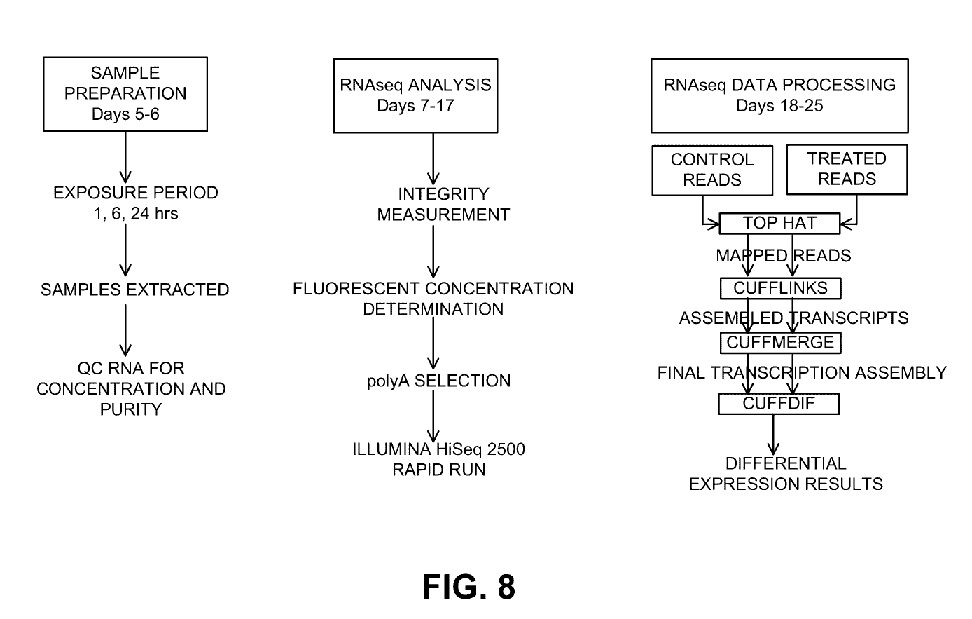

FIG. 8 provides a scheme showing a method for carrying out a transcriptomic analysis. See Trapnell et al., Nature Protocols 7(3), 562-578 (2012) for additional information on transcriptomic analysis. The transcriptome is the set of all messenger RNA molecules in one cell or a population of cells, and in many ways represents a precursor to the proteome. The main method for measuring the transcriptome of one or more cells is RNA-seq, though DNA microarrays can also be used to measure a transcriptome. RNA-seq (RNA sequencing), also called whole transcriptome shotgun sequencing, uses next-generation sequencing to reveal the presence and quantity of RNA in a biological sample at a given moment in time. Kits of carrying out RNA-seq are commercially available, and the methods are known to those skilled in the art. For a review of RNA-seq, see Wang et al., Nature Reviews Genetics 10 (1): 57-63 (2009).

### Mass Spectrometry

A “mass spectrometer” is an analytical instrument that can be used to determine the molecular weights of various substances, such as proteins and nucleic acids. It can also be used in some applications, e.g., to determine the sequence of protein molecules and the chemical composition of virtually any material. Typically, a mass spectrometer comprises four parts: a sample inlet, an ionization source, a mass analyzer, and a detector. A sample is optionally introduced via various types of inlets, e.g., solid probe, GC, or LC, in gas, liquid, or solid phase. The sample is then typically ionized in the ionization source to form one or more ions. The resulting ions are introduced into and manipulated by the mass analyzer. Surviving ions are detected based on mass to charge ratio. In one embodiment, the mass spectrometer bombards the substance under investigation with an electron beam and quantitatively records the result as a spectrum of positive and negative ion fragments. Separation of the ion fragments is on the basis of mass to charge ratio of the ions. If all the ions are singly charged, this separation is essentially based on mass.

In some aspects, the multi-omics analysis includes desorption ionization mass spectrometry analysis. Examples of desorption ionization mass spectrometry include mass spectrometry electrospray ionization (ESI) followed by tandem MS (MS/MS), liquid extraction surface analysis mass spectrometry (LESA-MS), and electrospray and desorption ionization methods (DESI).

### A. ESI

ESI is a convenient ionization technique developed by Fenn and colleagues (Fenn et al., Science, 246(4926):64-71, 1989) that is used to produce gaseous ions from highly polar, mostly nonvolatile biomolecules, including lipids. The sample is injected as a liquid at low flow rates (1-10 □L/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. Kabarle et al., Anal. Chem. 65(20):972A-986A (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer is delivered to tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; 6,756,586, 5,572,023 and 5,986,258.

### B. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., Anal. Chem., 74:2446, 2000) and bioactive peptides (Desiderio et al., Biopolymers, 40:257, 1996). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Bucknall et al., J. Am. Soc. Mass Spectrometry, 13(9):1015-27 (2002). Protein quantification has been achieved by quantifying tryptic peptides. Mirgorodskaya et al., Rapid Commun. Mass Spectrom., 14:1226, 2000. Complex mixtures such as crude extracts can be analyzed, but in some instances sample cleanup is required. Gobom et al., Anal. Chem. 72:3320, 2000. Desporption electrospray is a new associated technique for sample surface analysis.

### C. LESA

Liquid extraction surface analysis mass spectrometry (LESA-MS) is a surface profiling technique that combines micro-liquid extraction from a solid surface with nano-electrospray mass spectrometry. See Eikel et al., Rapid Commun Mass Spectrom. 25(23):3587-96 (2011), which evaluates LESA-MS by examining the distribution and biotransformation of unlabeled terfenadine in mice and comparing the findings to QWBA, whole tissue LC/MS/MS and MALDI-MSI. The spatial resolution of LESA-MS can be optimized to about 1 mm on tissues such as brain, liver and kidney, also enabling drug profiling within a single organ.

### D. DESI

DESI is a combination of electrospray (ESI) and desorption (DI) ionization methods. Ionization takes place by directing an electrically charged mist to the sample surface that is a few millimeters away. The electrospray mist is pneumatically directed at the sample where subsequent splashed droplets carry desorbed, ionized analytes. After ionization, the ions travel through air into the atmospheric pressure interface which is connected to the mass spectrometer. DESI-MS offers a number of advantages over traditional MS approaches, including (1) minimal sample preparation; (2) sample maintenance under ambient conditions outside the vacuum system; (3) rapid, high-throughput analysis; (4) the ability for in situ detection; and (5) label-free chemical imaging with basic instrumentation requirements. See Ifa et al., Analyst. 135:669-681 (2010).

### E. MALDI-MS

In some examples, the multi-omics analysis comprises matrix-assisted laser desorption/ionization (MALDI) mass spectrometry analysis. Since its inception and commercial availability, the versatility of MALDI-MS has been demonstrated convincingly by its extensive use for qualitative analysis. For example, MALDI-MS has been employed for the characterization of synthetic polymers, peptide and protein analysis (Zaluzec et al., Protein Expr. Purif., 6:109, 1995; Roepstorff et al., EXS, 88:81, 2000), DNA and oligonucleotide sequencing, and the characterization of recombinant proteins. Recently, applications of MALDI-MS have been extended to include the direct analysis of biological tissues and single cell organisms with the aim of characterizing endogenous peptide and protein constituents. Li et al., Trends Biotechnol., 18:151 (2000); Caprioli et al., Anal. Chem., 69:4751 (1997).

The properties that make MALDI-MS a popular qualitative tool—its ability to analyze molecules across an extensive mass range, high sensitivity, minimal sample preparation and rapid analysis times—also make it a potentially useful quantitative tool. MALDI-MS also enables non-volatile and thermally labile molecules to be analyzed with relative ease. It is therefore prudent to explore the potential of MALDI-MS for quantitative analysis in clinical settings, for toxicological screenings, as well as for environmental analysis. In addition, the application of MALDI-MS to the quantification of polypeptides (i.e., peptides and proteins) is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-MS applications, there are many problems inherent to the MALDI ionization process that have restricted its widespread use. Wang et al., J. Agric. Food. Chem., 48:3330 (2000); Desiderio et al., Biopolymers, 40:257 (1996). These limitations primarily stem from factors such as the sample/matrix heterogeneity, which are believed to contribute to the large variability in observed signal intensities for analytes, the limited dynamic range due to detector saturation, and difficulties associated with coupling MALDI-MS to on-line separation techniques such as liquid chromatography. Combined, these factors are thought to compromise the accuracy, precision, and utility with which quantitative determinations can be made.

In one example, the MALDI is high dynamic range MALDI (HDR-MALDI), which can maximize the sensitivity and dynamic range of mass spectrometric measurements. Spraggins et al., “Massively enhanced sensitivity and dynamic range for molecular imaging by High Dynamic Range (HDR)—MALDI FT-ICR MS. In 60^{th} American Society of Mass Spectrometry Conference, 2012, Vancouver, British Columbus, Canada. For instruments that have multiple ion traps prior to the mass analyzer, such as the 9.4 T Fourier transform ion cyclotron resonance (FTICR) MS and the proposed Orbitrap, ion signals can be enriched significantly by isolating a single ion, or m/z range, and accumulating and storing that ion population prior to detection. The ability to store ions is critical because it allows ions from many laser shots to be built up, resulting in dramatically increased sensitivity and dynamic range for a selected m/z window. This approach can increase signal intensities 2 to 3 orders of magnitude for selecting mass windows using our current FTICR MS. HDR MALDI experiments may also be performed by collecting multiple enriched m/z windows that span the entirety of the desired m/z range. The degree of signal enrichment for each window is determined by the total number of laser shots that are trapped in the accumulation ion trap prior to transfer to the mass analyzer. The resulting series of spectra that span the full mass range are then stitched together in a single full HDR-MALDI spectrum. In initial experiments, it was shown that HDR-MALDI FTICR MS spectra collected from kidney tissue sections resulted in ˜8,000 detectable ions of S/N>3 over a target mass range from m/z 500-1500. The same experiment performed on MALDI TOF or traditional full-scan FTICR resulted in ˜180 and ˜3000 ions, respectively. Each m/z window can be individually tuned to maximize performance, allowing ions to be measured from molecular species that are below the limit of detection for conventional instruments. For example, using HDR MALDI MS, ˜1×10⁻²² moles/cell (80 molecules/cell) of compounds of MW 1000 were detected over a 5×5 nm area.

In many MS applications, a matrix is used. The matrix chosen will depend on the particular mass spectrometry technique used to analyze the animal tissue. For example, in the case of MALDI-MS, matrix materials are typically solid organic acids. MALDI matrix materials include crystallized molecules such as 3,5-dimethoxy-4-hydroxycinnamic acid (sinapinic acid), α-cyano-4-hydroxycinnamic acid (CHCA) and 2,5-dihydroxybenzoic acid (DHB). Other MALDI matrix materials include 4-hydroxy-3-methoxycinnamic acid (ferulic acid), picolinic acid, and 3-hydroxy picolinic acid. A solution of one of these molecules is made, often in a mixture of highly purified water and an organic solvent such as acetonitrile (ACN) or ethanol. The matrix can be used to tune the mass spectrometer to ionize the sample in different ways. Acid-base-like reactions are often utilized to ionize the sample; however, molecules with conjugated pi systems, such as naphthalene like compounds, can also serve as an electron acceptor and thus a matrix for MALDI. The matrix density can also have an impact on matrix performance. Accordingly, in some embodiments, the matrix has a density from 0.3 to 3.0 mg/cm².

The matrix can be formed on the analytic substrate using a variety of methods known to those skilled in the art. For example, the matrix may be applied to the analytic substrate as an aerosolized mixture that is spray-coated onto the slide, either manually or using a robotic sprayer. Preferably, the matrix is applied as a substantially homogenous thin layer, covering an area corresponding roughly in size to the size of the animal tissue specimen.

The matrix used for carrying out MALDI-MS can vary between being an acidic (i.e., crystallized) matrix and an ionic matrix. The ionic matrix is a more liquid, permeable form, whereas the crystallized form provides a better substrate for mass spectrometry. Accordingly, in some embodiments, additional steps are carried out to convert the matrix from one form to another during the analysis method. For example, in some embodiments, after the animal tissue specimen has been placed on the matrix surface, the animal tissue specimen is then hydrated to facilitate interaction between the matrix and the animal tissue specimen. In some embodiments, the animal tissue specimen is hydrated for 5 to 10 minutes. However, after hydration, the matrix is then crystallized before conducting mass spectrometry to provide a better mass spectrometry substrate. Hydration can also include introduction of diisopropylethylamine or a similar compound to convert the matrix into ionic form, while crystallization can include introduction of a strong acid such as trifluoroacetic acid to convert the matrix to an acidic form.

In some aspects, the rapid phenotyping is carried out using advanced laser-based mass spectrometry (MS) for molecular profiling of the cellular responses of animal cells to chemical agents. Each individual measurement using a laser beam diameter of 50 to 100 □m contains 5-25 cells (assuming a 20 □m cell diameter), allowing the high sensitivity and high-throughput elucidation of proteins, lipids, and metabolites over the entire field of cells grown in wells or on a microscope slide. Data from thousands of ablated spots can be summed to provide maximum signal-to-noise (s/n) measurements with high statistical significance from a cell field that was treated homogenously with the chemical agent. In experiments involving a spatial-temporal gradient exposure, the data set is used to produce images or maps of the biomolecules over a range of exposure times and concentrations. Each ablated spot would be a pixel in images that are produced by plotting the relative intensity of any m/z peak in the spectra over the entire array of ablated spots. Cellular features (nuclei, membranes, and cytoplasm) can be separated and analyzed using well-established centrifugation methods prior to MS analysis. In addition, the cell field can be scanned with a laser beam of 2-5 □m to interrogate substructures in multiple cells (cell membranes, cytoplasm, and nuclei) through biocomputational “unmixing” of spectra to map biomolecules to the same substructures.

### Animal Cells

The animal cells 11 can be obtained from any animal, and any specific organ of an animal, and also include cell cultures which originated from an animal cell but have been cultured over time. Animal cells can be obtained from vertebrate animals and invertebrate animals. In some embodiments, the animal cells are obtained from a mammal, such as a domesticated farm animal (e.g., cow, horse, pig) or pet (e.g., dog, cat). In some embodiments, the animal cells are human cells. The animal cells can be present as part of an animal tissue specimen, or can be present as part of an animal cell culture.

In some examples, the animal cells 11 are part of an animal cell culture. Animal cell culture refers to the culturing of cells derived from animal cells. Cells can be isolated from tissues for ex vivo culture in several ways. Cells can be easily purified from blood or released from soft tissues by enzymatic digestion with enzymes such as collagenase, trypsin, or pronase, which break down the extracellular matrix. Alternatively, pieces of tissue can be placed in growth media, and the cells that grow out are available for culture. The temperature, gas mixture, and cell medium required for maintaining animal cells in culture are known to those skilled in the art. Animal cells can be grown either in suspension or adherent cultures. In some embodiments, the animal tissue sample is provided as an “organ on a chip,” which is a multi-channel 3-D microfluidic cell culture chip that simulates the activities, mechanics and physiological response of entire organs and organ systems. See for example M. Moyer, “Organs-on-a-Chip for Faster Drug Development”, Scientific American 25 Feb. 2011.

In some examples, the animal cells 11 are part of an animal tissue specimen. Intact tissue samples are obtained by standard methodologies for use as animal tissue specimens. In some embodiments, the tissue specimen has a thickness ranging from about 1 □□m to about 50 □□m, while in other embodiments the tissue specimen has a thickness ranging from about 3 □m to about 10 □m. In some embodiments, the animal tissue specimen is cryosectioned (i.e., frozen) animal tissue, which can be prepared using a cryostat. In other examples, the animal tissue specimen has been treated with chemical fixatives to preserve the tissue. Examples of chemical fixatives include formalin (e.g., 4% formaldehyde in phosphate buffered saline) and glutaraldehyde. These fixatives preserve tissues or cells mainly by irreversibly cross-linking proteins. Examples of other fixatives are osmium tetroxide or uranyl acetate. Tissue samples can also include paraffin wax, which is used after water has been removed from tissues to provide a medium that solidifies to allow thin sections of tissue to easily be cut. Accordingly, in some embodiments, the animal tissue specimen is formalin-fixed paraffin-embedded tissue.

Any type of animal tissue can be analyzed. The animal tissue evaluated can be healthy tissue, or it can be tissue that is diseased or injured. Examples of animal tissue suitable for evaluation include heart tissue, liver tissue, kidney tissue, prostate tissue, breast tissue, ovary tissue, uterine tissue, skin tissue, lung tissue, brain tissue, colon tissue, pancreatic tissue, and muscle tissue.

Biopsy procedures for obtaining the specimen will generally involve the sterility required of surgical operations, even though the tissues being sample are from cadavers or animals that will be sacrificed. For internal tissues, incisions will be made proximal to the tissue of interest, followed by retraction, excision of tissue and surgical closing of the incision. Superficial tissue sites are accessed by simple excision of the available tissue.

In some examples, an extraction step is carried out to extract the biomolecules from the animal 11 cells in order to provide an extract for analysis by mass spectrometry. For example, in some embodiments, peptides are extracted from an animal tissue specimen using solvent extraction prior to analyzing the partially digested animal tissue specimen by mass spectrometry. The extraction step can include an organic extraction and/or an aqueous extraction. Extraction will shrink and swell the matrix and tissue sample in order to release biomolecules (e.g., peptides) within the digested mixture, and can also separate the polypeptides, which migrate to the aqueous phase, from other components in the extraction mixture.

Another aspect provides a system for characterizing the cellular response of one or more animal cells to a chemical agent, which can include a computing device 12 comprising: a non-transitory memory 14 storing computer-executable instructions; and a processor 13 that executes the computer-executable instructions to at least: receive data generated by a multi-omic analysis representing an altered molecular phenotype of one or more animal cells 11 after exposure to a chemical agent 17; compare the data representing the altered molecular phenotype with data representing the normal molecular phenotype of the one or more animal cells; and output a characterization of the cellular response of the one or more animal cells 11 to the chemical agent 17 based on the results of comparing the data. In some examples, the testing apparatus 10 of the system further comprising a matrix-assisted laser desorption/ionization (MALDI) mass spectrometer for generating data representing an altered molecular phenotype of one or more animal cells 11 after exposure to a chemical agent 17.

### Testing Apparatus for Animal Cells and High-Throughput Analysis

A system for characterizing the cellular response of one or more animal cells to a chemical agent can also include a testing apparatus 10 for exposing one or more animal cells 11 to a chemical agent 17. The testing apparatus 10 can include any apparatus suitable for the type of animal cells. In the case of an animal tissue sample, the apparatus can be any substrate suitable for holding the animal tissue sample, such as a microscope slide. In other embodiments, and in particular when the animal cells are part of an animal cell culture, the apparatus is a well plate having a plurality of wells. Well plates are flat substrates typically made from inert plastic such as polystyrene that include a plurality of wells suitable for holding animal cells in culture. Well plates having a plurality of wells are commercially available, and typically include 6, 12, 24, 48, 96, or 384 wells that are regularly positioned throughout the area of the plate. The animal cells in the apparatus can be exposed to the chemical agent through any suitable means that result in the chemical agent contacting the animal cells, such as use of a pipette or fluidic channel.

In some aspects, the testing apparatus 10 can automate one or more steps of the method for the rapid analysis of a large number of samples. For example, the step of applying the protease and/or the matrix to the analytic substrate can be done robotically. Likewise, removing the digested mixture and extracting the polypeptides can be done robotically. Commercially available mass spectrometry system (e.g., MALDI instruments) can record the mass spectra of all the extract samples in quick succession. The analysis of the data can also be automated by employing a computer program to analyze generated data. With sufficient automation, a single person, with access to a MALDI instrument could use the automated techniques to measure as many as 1000 samples per day.

In some examples, the one or more of the animal cells 11 are placed in a regular column and row pattern (e.g., corresponding to that found in a standard 96 well plate) in a highly automated fashion, thereby ensuring that the rate of screening is dependent only on the speed of sequential analysis of the mass spectrometer. An automatic sampler can be used to transport samples between the purification system (which includes extraction and/or column purification) and the mass spectrometer. Autosamplers can be purchased from standard laboratory equipment suppliers such as Gilson and CTC Analytics. Such samplers function at rates of about 10 seconds/sample to about 1 min/sample. In some aspects, a computer and software are operably coupled to the apparatus for recording and analyzing mass spectrometer data and for controlling the automatic sampler.

In some examples, the method is a high-throughput method, or the system includes an apparatus enabling use of a high-throughput method. “High throughput mass spectrometry” is used herein to refer to a mass spectrometry system that is capable of analyzing samples at a rate of from about 100 or 200 samples per day to about 15,000 samples per day. In general, mass spectrometry and MALDI-MS in particular have proven to be highly amenable to high throughput applications in both clinical and basic research settings. For example, Sequenom Inc. (San Diego, Calif.) has established MALDI-MS as an effective technique in the field of genotype profiling, and is providing diagnostic products in this area. In some embodiments, the method is capable of analyzing about 200 samples in less than an hour, e.g., 200 samples are injected into a mass spectrometer and analyzed in less than an hour. High throughput screening preferably takes advantage of the ability to automate the data acquisition and data analysis methods. In some examples, the testing apparatus 10 comprises a high throughput apparatus including a robot arm for the rapid analysis of a plurality of wells.

### Visualization of the Cellular Response

A system for characterizing the cellular response of one or more animal cells to a chemical agent can also include a display device 15. The display device 15 can output a characterization determined by a computing device 12 based on a comparison between input altered data and normal data. For example, the output can include a visualization of the characterization and the display device 15 can be a graphical display device (such as a computer monitor or other graphical user interface, for example). In another example, the output can include an audio portion and the display device 15 can include an audio output device (e.g., speakers).

In some examples, the visualization can include one or more images. For example, the method of analyzing animal tissue also includes imaging the partially digested animal tissue specimen that has been analyzed. Once the levels of the one or more biomolecules have been determined, they can be displayed in a variety of ways. For example, the biomolecule levels can be displayed graphically on the display device 15 as numeric values or proportional bars (i.e., a bar graph) or any other display method known to those skilled in the art. The graphical display can provide a visual representation of the amounts of the one or more biomolecules in the tissue specimens being evaluated. In addition, the display device 15 can also be configured to display a comparison of the levels of the one or more biomolecules to corresponding control values.

Imaging can also include more sophisticated representations of biomolecule levels. In the context of Mass Spectrometry, imaging refers to techniques used to visualize the spatial distribution of biomolecules by their molecular masses using a two-dimensional map of the animal tissue specimen. The image is generated by taking the data obtained through mass spectrometric analysis of the animal tissue specimen, running it through software, and displaying the resulting image using an the display device 15, such as an optical display device, like a flat panel liquid-crystal, plasma, or light-emitting diode display.

This disclosure is illustrated by the following examples. It is to be understood that the particular examples, materials, amounts, and procedures are to be interpreted broadly in accordance with the scope and spirit of the invention as set forth herein.

### EXAMPLES

### Example 1

### Global Cell Response Captured in 30 Days Yields Comprehensive Drug Mechanism

This experiment shows the utility of a platform resource that empirically derives a global mechanism of action (MOA) (e.g., identifying pathways de novo) for a compound in less than 30 days. The platform utilizes multi-omics technologies for large scale measurement of molecular events to generate a comprehensive picture of the cellular response to an exogenous compound. The comprehensive picture includes identification of pathways of the MOA. Additionally, this integrated, analytical, computational, and high throughput platform deduces dose conditions for unknown compounds and integrates cell-based measurements, such as apoptosis, with multi-omics data, facilitating a comprehensive analysis of cellular responses with temporal resolution. Network analysis identifies associations that are not detected by reductionist approaches. Importantly, this platform enables discovery of cellular processes outside of targeted pathways, providing molecular information about pleiotropic responses. By using network analysis, this platform captures pathways without directly measuring all pathway members.

Proof-of-principle for this platform resource is demonstrated using the chemotherapeutic agent cisplatin. Using cisplatin, it was demonstrated that this platform can identify primary MOA and pathways important for side effects and resistance. Research over the past 20 years establishes a few dozen compounds implicated in cisplatin's primary MOA. In 30 days this platform quantified over 10,000 unique molecular changes, including 55% of the species in an expanded canonical network. Importantly, the data captured novel pathways that may inform clinical observations of cisplatin resistance. A driving aim for this technology is to move beyond the limits of targeted analyses informed by established pathways and to provide a resource for accelerated understanding of MOA.

This platform provides several key developments in MOA determination. First, a 3-day screening platform determines relevant exposure and dose, using MS to determine the maximal molecular changes. Second, comprehensive molecular data are collected within 2-3 weeks, including PTMs and metabolomics. These data can generate a tunable output of the final network or mechanism based on statistical confidence in empirical measurements. Last, this platform provides high throughput, comprehensive MOA assessment. Previous studies successfully identified compound MOA from published datasets of transcriptional changes in response to compounds. di Bernardo et al., Biotechnol. 23, 377-383 (2005); Woo et al., Cell 162, 441-451 (2015). This platform collects post-transcriptional and post-translational data to capture MOA beyond gene regulation. Apart from these enhancements, the analysis of cisplatin demonstrates that data acquired with this platform provide nearly complete confirmation of the primary MOA for cisplatin cytotoxicity. Furthermore, the data contribute to a more complete description of the biological processes potentially involved in cisplatin resistance. The results underscore how an integrated omics approach drives the generation of testable hypotheses that directly relate to global cellular responses.

### Experimental Procedures

Cell Culture

A549 cells were cultured in DMEM or SILAC DMEM (Thermo Scientific) and treated with 50 μM cisplatin (Tocris Bioscience) or ddH2O.

Screening

Cell viability and apoptosis were assessed by CellTiter Glo (Promega) and ApoONE (Promega) kits, respectively. Molecular changes were screened using a MALDI524 FTICR MS platform.

Multi-Omics Analysis

Samples were analyzed for transcriptome changes by RNA sequencing at the Genomics Services Lab, HudsonAlpha, for proteome changes by label free, SILAC, and phospho-enriched SILAC LC-MS/MS, and metabolome changes by UPLC-IM-MS and data-independent acquisition (MSE) using both hydrophilic-interaction liquid chromatography and reverse phase liquid chromatography.

Computational Analysis and Data Mining

Data from all platforms were integrated and parsed for significantly changed, unique species. An analysis pipeline was developed implemented in the Python programming language as part of the PySB modeling framework. Lopez et al., Mol. Syst. Biol. 9, 646 (2013). Bioservices (Cokelaer et al., Bioinforma. Oxf. Engl. 29, 3241-3242 (2013)) was used to download pathways from the KEGG database (Kanehisa et al., Nucleic Acids Res. 40, D109-D114 (2012)) that contain any proteins from a list of seed species. These pathways were combined to form a unified network based on common protein species. To examine the species-to species interactions in our data networks, the open source systems biology platform Cytoscape (Shannon et al., Genome Res. 13, 2498-2504 (2003)), the QIAGEN IPA network analysis tool, and annotated literature were used.

Cell Culture

Human lung carcinoma A549 cells were obtained from ATCC (Manassas, Va.) and cultured in Dulbecco's Modified Eagle's Medium (DMEM, Gibco) with 10% v/v heat-inactivated fetal bovine serum (Atlanta Biologicals) and 1% v/v penicillin/streptomycin (P/S) (Gibco) at 37° C. with 5% CO2 atmosphere. Stable isotope labeling by amino acids in cell culture (SILAC) media were prepared using SILAC DMEM (Thermo Scientific) containing 10% v/v dialyzed heat-inactivated FBS (Fisher), 1% v/v P/S, proline (250 mg/L) (Thermo Scientific), and appropriate light and heavy labeled arginine (84 mg/L), lysine (146 mg/L). Cells were seeded to tissue-culture treated vessels at the following densities: 96-well plates, 1×104 cells per well in 95 μL; 24-well plates, 5×104 cells per well in 475 μL; 6-well plates, 2×105 cells per well in 3.1 mL; 100 mm dishes, 2×105 cells per dish in 10 mL; 100 mm flasks, 4×106 cells per flask in 20 mL. In all experiments, cells were cultured for 24 h prior to treatment with cisplatin or no treatment control.

For proteomic and metabolomic label-free analyses, five sterile indium tin oxide (ITO)-coated glass slides (Delta Technologies) were placed in a tissue culture flask with removable lid (TPP). Cisplatin (Tocris Bioscience) was added to a final concentration of 50 μM from a 4 mM stock. For proteomic analyses, slides were removed and washed 3 times with PBS, placed on dry ice, flash-frozen in liquid nitrogen, and stored at −80° C. prior to lysis. For metabolomics analysis, slides were washed in 50 mM ammonium formate.

For SILAC experiments, A549 cells were cultured in SILAC media for two weeks to ensure isotopic amino acid incorporation (verified by proteomic analysis). Cells were plated to 10 cm dishes and incubated with or without 50 μM cisplatin. Following treatment cells were washed 3 times with 4° C. PBS. For phospho-SILAC, cells were washed with 4° C. PBS containing 1 mM sodium orthovanadate.

Cell Viability and Apoptosis Assays

Cisplatin (Tocris Bioscience) was serially diluted in water to generate a 20× stock plate representing the full spectrum of concentrations tested; 5 μL was applied per well containing 95 μL of cells in black-welled 96-well plates (Costar). At each time point, cell viability and apoptosis were assessed by CellTiter-Glo (Promega) and ApoONE (Promega) kits, respectively. Plates were incubated at 37° C. in a plate reader (BioTek) with data acquisition every 10 min by luminescence (1 s read time, 200 gain) and fluorescence (485 nm excitation, 528 nm emission). Triton X-100 (Research Products International Corp) and staurosporine (Tocris Bioscience) were added as positive controls for CellTiter-Glo and ApoONE, respectively. Each assay was performed in triplicate wells in triplicate plates. Cell viability was also assessed using the LIVE/DEAD Viability/Cytotoxicity Kit for mammalian cells (ThermoFisher). Probes were added to the cells for 30 minutes at room temperature, and images were acquired using the fluorescein and rhodamine filter sets on the Olympus IX73.

Molecular Screening Assay

Cells were seeded in 24-well plates and left untreated or were treated with cisplatin from 20× stocks. After incubation for the respective times, wells were washed 3 times with PBS and stored at −80° C. The effect of different toxin dosage amounts and exposure times on the molecular content of a sample may be evaluated and reported directly for thousands of molecular species based upon the mass spectra that were collected for each of the samples. A comparison of the mass-to-charge (m/z) peak content between spectra from dosed cells and a reference spectrum from a non-dosed control sample delivers a mass spectrometric assessment of toxicity-induced molecular variation. However, seeking a data-driven global comparison instead of focusing on specific molecular species of interest makes the assessment of molecular change between the different experimental conditions very complex and high dimensional. To determine which dosage amount induces the greatest molecular change, a quick and broad assessment of toxicity-induced molecular variance summarizing the entire recorded mass range was developed. This approach projects the high-dimensional variance between the spectra of different experimental conditions to a lower dimensional representation, which allows the researcher to instantly assess overall molecular variance between different dosage conditions as a single scalar value.

Samples for rapid molecular screening analysis were prepared within the same 24-well cell culture plates in which they had been grown. Cells were first lysed with 100 μL of 25% methanol (MeOH) followed by reduction with 1 μL of 40 mM dithiothreitol (DTT) for 30 minutes at 37° C. Alkylation was performed with 2 μL of 80 mM iodoacetamide (IAA) for 30 minutes at room temperature in the dark. Subsequently, proteins were digested using 1 μL of 1 μg/μL Trypsin Gold, MS grade (Promega) for 3.5 h at 37° C. Trypsin was inactivated and digestion was stopped using 2 μL of 0.5% acetic acid. Protein digests were then transferred to PCR tubes and centrifuged to remove cell debris. Sample were desalted (EMD Millipore ZipTip, C18) and 300 nL of the cleaned up protein digests were spotted onto a MALDI anchor plate (Bruker) along with 300 nL of 10 mg/57 mL α-cyano-4-hydroxycinnamic acid (CHCA) in 50% acetonitrile (ACN), 0.1% trifluoroacetic acid (TFA). Screening analysis was performed using a Bruker 15T solariX MALDI FTICR mass spectrometer (Bruker Daltonics, Billerica, Mass., USA), which has mass resolution >100,000 and mass accuracy <2 ppm. The instrument is equipped with an Apollo II dual MALDI/ESI ion source and a Smartbeam II 2 kHz Nd:YAG (355 nm) laser. All data were collected using the small laser setting (˜50 μm) with the instrument set to randomly “walk” between scans within each MALDI sample spot. Acquisition was <30 seconds/sample. Data were collected from m/z 400-5,000 with a resolving power of ˜85,000 at m/z 1,000. Special tuning of the Funnel RF amplitude (190 Vpp), accumulation hexapole (1.4 MHz, 1200 Vpp), transfer optics (2 MHz, 305 Vpp), time of flight delay (1.5 ms), and ICR cell (sweep excitation power: 21%) were required for peptide analysis. External calibration was performed prior to analysis using cesium iodide (CsI) clusters. DataAnalysis 4.2 (Bruker Daltonics, Billerica, Mass., USA) was used to export spectra into a form compatible with custom data processing scripts (standard XY ASCII).

Within each time point (represented by a 24-well plate), seven different dosage conditions were measured: six different toxin dosages and one reference condition (e.g., untreated). Per dosage condition there were nine distinct replicate measurements made: each dosage condition was represented by three replicate wells, from which three technical replicate spectra were measured per well. As a result, for each time point, seven dosage conditions with nine replicates amount to the acquisition of 63 MALDI FTICR mass spectra per 24-well plate. These spectra themselves are an average of 10 individual spectra that were acquired at random locations within a single MALDI spot, with acquisition at each location accumulating 500 laser shots per spectrum. The screening method first trimmed these 63 spectra to the relevant mass range of m/z 900 to 5000, yielding a data matrix of 63 rows×381,513 columns. Each row captures a spectrum and each column reports a particular m/z bin across all spectra in this table. To ensure robustness against outlier measurements, the nine technical replicates per dosage condition were first normalized to each other and then summarized into a single representative spectrum for that dosage condition by taking the median spectrum across all nine normalized spectra. This step employed standard total ion current (TIC) based normalization (Rasmussen and Isenhour, J. Chem. Inf. Comput. Sci. 19, 179-186 (1979)), implemented in R using the R Core Team 2012 procedure. The resulting 7×381,513 data matrix contained a single consensus mass spectrum for each of the dosage conditions. The next step prepared the different dosage conditions for direct comparison by normalizing the seven spectra to each other, using the same TIC based normalization method that was applied across replicates in the previous step. The seven normalized spectra in the resulting 7×381,513 data matrix were now projected onto the same ion intensity scale, allowing for direct comparison of intensity values. However, these spectra described the full profile across the entire mass range, whereas the screening method should focus only on the detected ion species and their peaks. Therefore, the spectra were translated into peak lists and their corresponding peak heights. To accomplish this, an R script mined peak locations along the m/z axis from an average spectrum of all seven spectra, recording any peak that surpassed a threshold of 1.7% of the highest recorded peak intensity. In all time points, this threshold returned ˜4,000 individual ion peaks, ensuring a broad spread of contributing ion species into our overall molecular variance assessment. Retrieving peak intensities from the seven full profile spectra for each of these peak locations delivered a 7×˜4,000 data matrix of peak intensities, which reports molecular content across different ion species for each of the seven dosage conditions. The final step of the screening procedure is to cast the ˜4,000-dimensional difference between two such rows into a single summary ‘molecular change’ value without losing too much information. The first dimensionality reduction step uses principal component analysis (PCA) (Jolliffe, Principal Component Analysis (New York: Springer-Verlag) 2002) to project the seven measurements from a ˜4,000-dimensional space (of which many dimensions are correlated) to a two-dimensional space in which the two dimensions are orthogonal and the axes represent the directions of highest variance. The second step is to calculate the distance between each pair of measurements in this two-dimensional space using the ‘cosine’ distance measure, which is actually one minus the cosine of the included angle between the measurement vectors. These steps are accomplished in MATLAB (The Mathworks Inc., Natick, Mass.) using the ‘princomp’ function for PCA projection and the ‘pdist’ function for the cosine distance calculation. The screening method then reports back the distance value between the reference measurement and each of the particular dosage measurements. These six distance values can be considered a representation of the molecular difference between a dosage measurement and the reference measurement, effectively reporting a relative measure for molecular variance for each dosage amount. Within a set of seven dosage conditions, these values show which dosage amount results in stronger molecular deviation from the reference. The molecular variance score reduces the information to a single objectively measured value that represents the magnitude of the perturbation induced by cisplatin comparable among all the dosing conditions tested.

Transcriptomics

Cells were seeded in 6-well plates and incubated with or without 50 μM cisplatin for 1 h, 6 h, or 24 h. RNA was isolated using the RNeasy Mini Kit (Qiagen). For each time point, untreated and cisplatin-treated samples were isolated in triplicate and analyzed by the Genomics Services Lab at HudsonAlpha. RNAseq was performed using poly(A) selection on an Illumina HiSeq v4 sequencing platform. Reads were paired-end with a read length of 50 bp and 50 million reads per sample.

Proteomics Label Free LC-MS/MS

Cells were scraped from slides and lysed in 300 μL of 50 mM Tris pH 8, 150 mM NaCl, 1% Nonidet 40, 1 mM EDTA with added HALT Protease Inhibitor Cocktail (Thermo Scientific), centrifuged, and assayed for protein concentration (BCA Protein Assay, Thermo Scientific Pierce) using a SpectraMax M2e Microplate Reader with SoftMax Pro software version 5 (Molecular Devices). Aliquots of 100 μg of protein were acetone precipitated in six times the volume for 2 h at −80° C. Precipitates were washed three times with cold acetone and reconstituted in 10 μL of neat trifluoroethanol (TFE) and 10 μL of 100 mM Tris (pH 8.0). Samples were reduced with 1 μL of 0.5 M Tris(2-carboxyethyl)phosphine hydrochloride (TCEP) for 30 minutes at room temperature and alkylated with 2 μL of 0.5 M IAA for 30 minutes at room temperature in the dark. Samples were diluted with 100 mM Tris (pH 8.0) to obtain a final solution containing 10% TFE. The samples were digested with 2 μg of trypsin (a ratio of 50:1 protein to enzyme) overnight at 37° C. A solution of 60% formic acid (FA) was added to the samples until they reached pH 3. Aliquots of 5 μg of digested sample were desalted using C18 spin tips (Protea) according to the supplied protocol and dried samples were reconstituted in 15 μL of 0.1% FA. Five replicates of cisplatin treated and control (untreated) cells were prepared in parallel.

Samples were analyzed on a Thermo Scientific Orbitrap Fusion Tribrid mass spectrometer in line with a Thermo Scientific Easy-nLC 1000 UHPLC system. Samples, 2 μL, were injected via the autosampler and loaded onto a Thermo Scientific Acclaim PepMap 100 C18 UHPLC column (75 μm×250 mm, 2 μm particle size, 100 Å pore size), with 0.1% FA in water (mobile phase A). Peptides were separated over a 140 minute two-step gradient with initial conditions set to 100% mobile phase A for 5 minutes before ramping to 20% mobile phase B, 0.1% FA in ACN, over 100 minutes and then 32% mobile phase B over 20 minutes. The remainder of the gradient was spent washing at 95% mobile phase B and returning to initial conditions. Eluted peptides were ionized via positive mode nanoelectrospray ionization (nESI) using a Nanospray Flex ion source (Thermo Fisher Scientific). The mass spectrometer was operated using a 3 second top speed data-dependent acquisition mode. Fourier transform mass spectra (FTMS) were collected using 120,000 resolving power, an automated gain control (AGC) target of 200,000, and a maximum injection time of 50 ms. Precursor ions were filtered using monoisotopic precursor assignment according to charge state (9>z >1 required). Previously interrogated precursor ions were excluded using a 30 s dynamic window (±10 ppm). Precursor ions for tandem mass spectrometry (MS/MS) analysis were isolated using a 1.5 m/z quadrupole mass filter window. Precursor ions were fragmented via higher energy dissociation (HCD) using a normalized collision energy of 35%. Ion trap fragmentation spectra were acquired using an AGC target of 1,000 and maximum injection time of 40 ms. Data were analyzed via Protalizer (Vulcan Analytical Inc.) to identify proteins and determine a fold change in proteins common to the treated and control samples. Search parameters were set to include carbamidomethyl, phosphorylation, and oxidation modifications, as well as methionine-containing and miscleaved peptides (maximum of two miscleavages). Both peptide and protein target FDR rates were set to 1%. For the Orbitrap-LTQ, data precursor and fragment tolerances were 20 ppm and 0.6 Da respectively. For Orbitrap-Orbitrap data precursor and fragment ion tolerances were both 20 ppm. Changes in protein abundance were considered statistically significant at an absolute value of 1.5 or above and a p-value of ≦0.1.

SILAC LC-MS/MS

Cells grown in SILAC media (as described above) were lysed in 500 μL of 50 mM Tris pH 8, 150 mM NaCl, 1% Nonidet 40, 1 mM EDTA with added HALT Protease/Phosphatase Inhibitor Cocktail (Thermo Scientific), centrifuged at 15,871×g for 10 min at 4° C., and assayed as detailed above for protein concentration (BCA, Pierce). Aliquots of 50 μg of protein from heavy and light labeled cell lysates (representing control and treated exposures) were mixed 1:1. The combined lysate was then precipitated in six times the volume of ice-cold acetone overnight at −20° C. Following precipitation, samples were centrifuged at 18,000×g at 4° C., and precipitates were washed with cold acetone, dried, and reconstituted in 100 mM Tris pH 8, containing 50% TFE. Samples were digested as described for label-free samples. Two replicates of cisplatin treated and control cells were prepared per time point. Replicates represented a label-swap with one replicate as heavy-cisplatin treated, light-control and the other as light-cisplatin treated, heavy-control.

Samples were analyzed by LC-MS/MS on a Q Exactive mass spectrometer (Thermo Scientific) coupled to an Eksigent NanoLC. Peptides were loaded onto a self-packed biphasic C18/SCX MudPIT column using a Helium pressurized cell (pressure bomb). The MudPIT column consisted of 360×150 μm i.d. fused silica, which was fitted with a filter-end fitting (M-120, IDEX Health & Science) and packed with 6 cm of Luna SCX material (5 μm, 100 Å, Phenomenex) followed by 4 cm of Jupiter C18 material (5 μm, 300 Å, Phenomenex). Once the sample was loaded, the MudPIT column was connected using an M-520 microfilter union (IDEX Health & Science) to a laser-pulled emitter analytical column (360 μm×100 μm i.d.) packed with 20 cm of C18 reverse phase material (Jupiter, 3 μm, 300 Å, Phenomenex). MudPIT analysis was performed with an 11-step salt pulse gradient (25, 50, 75, 100, 150, 200, 250, 300, 500, 750, and 1000 mM ammonium acetate). Following each salt pulse, peptides were gradient-eluted from the reverse phase analytical column at a flow rate of 500 nL/minute, and the mobile phase solvents consisted of 0.1% FA in water (solvent A) and 0.1% FA in ACN (solvent B). A 120-mM reverse phase gradient was used consisting of 2-50% solvent B in 105 minutes followed by a 15 minute equilibration at 2% solvent B for the peptides from the first 10 SCX fractions. For the last fraction, the peptides were eluted from the reverse phase analytical column using a gradient of 2-98% solvent B in 105 minutes. Peptides were introduced into the mass spectrometer via nano electrospray ionization. The Q Exactive was operated in the data-dependent mode acquiring HCD MS/MS scans (R=17,500) after each MS1 scan (R=70,000) on the 20 most abundant ions using an MS1 ion target of 1×106 ions and an MS2 target of 1×105 ions. The maximum ion time for MS/MS scans was set to 100 ms, the HCD-normalized collision energy was set to 30, dynamic exclusion was set to 30 s, and peptide match and isotope exclusion were enabled.

Data were analyzed via MaxQuant software package, version 1.3.0.5 (Cox and Mann, Nat. Biotechnol. 26, 1367-1372 (2008)) to determine protein identification and fold change differences between common proteins in the treated and control samples. MS/MS spectra were searched against a human subset database created from the UniprotKB protein database. Precursor mass tolerance was set to 20 ppm for the first search, and for the main search, a 10-ppm precursor mass tolerance was used. The maximum precursor charge state was set to 7. Variable modifications included carbamidomethylation of cysteine (+57.0214) and oxidation of methionine (+15.9949). Enzyme specificity was set to Trypsin/P, and a maximum of two missed cleavages were allowed. The target-decoy false discovery rate (FDR) for peptide and protein identification was set to 1% for peptides and 1% for proteins. A multiplicity of 2 was used, and Arg10 and Lys8 heavy labels were selected. For SILAC protein ratios, a minimum of two unique peptides and a minimum ratio count of 2 were required, and the requantify option was enabled. Protein groups identified as reverse hits were removed from the datasets, along with non-human contaminants and identifications to which multiple proteins were assigned. To determine significance of fold change for the quantified proteins, the methods outlined in Thissen et al. (Thissen et al., J. Educ. Behav. Stat. 27, 77-83 (2002)) were followed. Briefly the mean and standard deviation were calculated for the log 2 value of the normalized heavy/light ratio. Next a p-value was calculated from the distribution of these log 2 values. The data were ranked by p-value and sorted in descending order. The Benjamini-Hochberg formula was applied and data with corresponding p-values less than the resulting Benjamini-Hochberg value were determined to have statistical significance. Protein groups containing non-human proteins and multiple proteins were excluded from further analysis. For networking and analysis purposes, a final step was performed to determine if proteins were considered significantly changed or unchanged. Proteins were considered significantly changed if they displayed a significant change in the same direction in both label-swapped replicates, if they displayed a significant change in one replicate and an insignificant change (fold change >|1.5|) in the same direction in the other replicate, or if they displayed a significant change and were found in only one replicate. Proteins that displayed a significant change in opposite directions in each replicate or displayed a significant change in one replicate but were unchanged in the other replicate were not considered significantly changed.

ph-SILAC LC-MS/MS

For phospho-enrichment SILAC cells grown in SILAC media were lysed in 500 μL of 50 mM Tris pH 8, 150 mM NaCl, 1% Nonidet 40, 1 mM EDTA, 1 mM PMSF, centrifuged at 15,871×g for 10 minutes at 4° C., and the supernatant was assayed for protein concentration. Aliquots of 900 μg of protein from heavy and light labeled cell lysates were mixed 1:1. To each sample was added an equal volume of neat TFE. Samples were reduced for 30 minutes at room temperature with 1 μL of 0.5 M TCEP per 100 μg of sample and then alkylated for 30 minutes at room temperature in the dark with 2 μL of 0.5 M IAA per 100 μg of sample. Samples were diluted with 100 mM Tris to a final volume of 10 times the amount of TFE added and the samples were digested with trypsin at a ratio of 25:1 protein to enzyme overnight at 37° C. TFA was added to the samples until they reached pH 3. Digested samples were desalted using Waters Sep-Pak Light C18 cartridges (130 mg) and a vacuum assisted solid-phase extraction manifold (Supelco). Samples were diluted with an equal volume of 0.1% TFA to dilute the concentration of TFE to below 5%. Columns were conditioned with 5 mL of 100% ACN and equilibrated with 3×5 mL of 0.1% TFA. Samples were loaded at 1-2 mL/min and washed with a volume of 0.1% TFA equal to the pre-diluted sample volume. Bound peptides were eluted at 1-2 mL/min with sequential 1 mL aliquots of 10%, 15%, 20%, 25%, 35%, 40%, and 60% ACN each containing 0.1% TFA. The eluate was divided among eight tubes and then dried at ambient temperature using a SpeedVac concentrator (Thermo Scientific).

Samples were enriched for phosphopeptides using TiO₂ beads. Two 30 mg aliquots of TiO₂ beads were washed three times with 300 mg/mL lactic acid in 80% ACN, 20% water, 0.05% heptafluorobutryic acid (HFBA). Each of the eight desalted dried fractions were reconstituted in 250 μL of 300 mg/mL lactic acid in 98% water, 2% ACN, 0.05% HFBA and combined; 1 mL of sample was incubated with each aliquot of TiO2 beads for 30 minutes with mixing (to bind phosphopeptides) and centrifuging before the supernatant was removed. The beads were washed with 500 μL of 80% ACN, 20% water, 0.05% HFBA for 5 minutes. The supernatant was removed, and the beads were washed two times for 5 minutes with 500 μL of 300 mg/mL lactic acid in 80% ACN, 20% water, 0.05% HFBA. Finally the beads were washed three times with 500 μL of 80% ACN, 20% water, 0.05% HFBA. Bound peptides were eluted into three fractions. The beads were first incubated with 500 μL of 0.5 M NH4OH for 5 minutes, and then incubated twice with 500 μl of 5 M NH4OH for 5 minutes. Samples were dried and each of the three fractions were reconstituted in 20 μL of 0.1% FA and the fractions combined prior to LC-MS/MS analysis. Two replicates of cisplatin treated and control cells were prepared. Replicates represented a label-swap with one replicate as cisplatin treated heavy, control-light and the other as cisplatin treated-light, control-heavy.

Samples were analyzed on a Linear Trap Quadrupole-Orbitrap Velos (Thermo Scientific) in line with an Eksigent NanoLC. Phosphopeptides were loaded on a MudPIT column as described above for SILAC peptides. An 8-step salt pulse gradient (25, 50, 75, 100, 150, 250, 500, and 1000 mM ammonium acetate) was performed. Following each salt pulse, peptides were gradient-eluted from the reverse phase analytical column at a flow rate of 500 nL/minute, and the mobile phase solvents consisted of 0.1% FA in water (solvent A) and 0.1% FA in ACN (solvent B). A 120-min reverse phase gradient was used that consisted of 2-40% solvent B in 105 min followed by a 15 min equilibration at 2% solvent B for the peptides from the first seven SCX fractions. For the last fraction, the peptides were eluted from the reverse phase analytical column using a gradient of 2-98% solvent B in 105 minutes. Peptides were introduced into the mass spectrometer via nano-electrospray ionization into the mass spectrometer and the data were collected using a 17-scan event data-dependent method. Full scan (m/z 350-2000) spectra were acquired with the Orbitrap as the mass analyzer (resolution, 60,000), and the 16 most abundant ions in each MS scan were selected for fragmentation in the Velos ion trap. An isolation width of 2 m/z, activation time of 10 ms, and 35% normalized collision energy were used to generate tandem mass spectrometry spectra.

The data were analyzed as described for SILAC data with two differences: 1) variable modifications also included phosphorylation of serine, threonine and tyrosine (+79.9663) and 2) the target-decoy false discovery rate (FDR) for identification was set to 1% for peptides and 2% for proteins. A minimum ratio count of 2 was required, and the requantify option was enabled. Prior to evaluating significance, all rows with missing H:L normalized values and reverse hits were removed. Significance of the fold change for peptides was determined as described above for SILAC proteins (Thissen et al., J. Educ. Behav. Stat. 27, 77-83 (2002)). All non-human hits were removed, and no additional filtering of phosphopeptides was performed. For networking and analysis purposes, a final step was performed to determine if proteins were considered significantly changed or unchanged. In cases where unique peptides had multiple hits, fold change values for all hits within a replicate (heavy or light treated) were averaged and the overall significance of the combined values evaluated as higher or lower than 50% true. If a peptide had a low percent true value (<50%) in both replicates it was considered unchanged. If a peptide had a high percent true value (>50%) in both replicates it was considered as follows: peptides were considered significantly changed if they 1) displayed a significant change in the same direction in both label-swapped replicates, 2) displayed a significant change in one replicate and an insignificant change (fold change >|1.5|) in the same direction in the other replicate, or 3) displayed a significant change and were found in only one replicate; peptides that displayed a significant change in opposite directions in each replicate or displayed a significant change in one replicate but were unchanged in the other replicate were not considered significantly changed. Significantly changed unphosphorylated peptides measured in the ph-SILAC experiments were grouped with the SILAC data.

Metabolomics

All solvents used for metabolite extraction and analysis (MeOH, H2O, ACN, FA, ammonium formate and ammonium acetate) were LC/MS grade (Fisher Scientific, Fair Lawn, N.J.). Cell slides (˜6-7×103 cells/slide) were kept at −80° C. or dry ice until ready for metabolomic sample processing. Intracellular metabolites were extracted by scraping individual cell slides in 350 μL of cooled (4° C.) 2:2:1 (v:v:v) ACN:MeOH:H2O. Individual samples were dried in vacuo just until dried and reconstituted in 1 mL of 75:25 (v:v) ACN:H2O (dry ice cooled), vortexed for 30 s, sonicated (five 1 s pulses at 30% amplitude while on ice) and incubated at −80° C. for 2 h. After incubation, samples were cleared by centrifugation at 15,000 rpm for 15 min, and the resulting supernatant was removed, halved in volume and evaporated to dryness in a vacuum concentrator. Dried extracts were reconstituted in 100 μL of reverse phase reconstitution solvent mixture containing 98:2 (v:v) H2O:ACN with 0.1% FA for reverse phase analysis or 100 μL of normal phase reconstitution solvent mixture containing 80:20 (v:v) ACN:H2O for normal phase analysis; followed by centrifugation for 60 s at 5,000 rpm to remove insoluble debris. Quality control samples were prepared by combining equal volumes (20 μL) of each sample type and samples were transferred to HPLC vials prior to IM-MS analysis.

Metabolomic Mass Spectrometry Analysis

UPLC-IM-MS and data-independent acquisition (MSE) were performed on a Synapt G2 HDMS (Waters Corporation, Milford, Mass.) mass spectrometer equipped with a nanoAcquity UPLC system and autosampler (Waters Corporation, Milford, Mass.). Chromatographic separations were achieved using both hydrophilic-interaction liquid chromatography (HILIC) and reverse phase liquid chromatography (RPLC). A 1.7 μm (1 mm×100 mm) ACQUITY BEH amide column (Waters Corporation) was used for HILIC analysis and reverse phase liquid chromatography was performed using a 1.8 μm (1 mm×100 mm) HSS T3 ACQUITY column fitted with a 1.8 μm HSS C18 pre-column (2.1 mm×5 mm). Samples were analyzed three times each in UPLC-HILIC-HDMSE and UPLC-RPLC-HDMSE in positive ionization mode. For HILIC analysis, mobile phase A was 9:1 (v:v) H2O:ACN and mobile phase B was 9:1 (v:v) ACN:H2O, both with 0.1% FA and 10 mM ammonium acetate. The following elution gradient was used for HILIC analysis: 0 min, 12.5% A; 1 min, 12.5% A; 4 min, 62.5% A; 10 min, 37.5% A; 11 min, 80% A; 13 min, 80% A; 14 min, 12.5% B. Flow rates for HILIC analysis were 90 μL/min with a column temperature at 30° C. and an injection volume of 5 μL. For RPLC analysis, mobile phase A was H2O and mobile phase B was ACN, both with 0.1% FA. The following elution gradient was used for RPLC analysis: 0 min, 99% A; 1 min, 99% A; 10 min, 40% A; 20 min, 99% A; 22 min, 99% A; 25 min, 1% A. Flow rates for RPLC analysis were 75 μL/min with a column temperature of 45° C. and an injection volume of 5 μL. HDMSE analyses were run using resolution mode, with a capillary voltage of 3 kV, source temperature at 120° C., sample cone voltage at 35V, source gas flow of 300 Ml min−1, desolvation gas temperature of 325° C., He cell flow of 180 mL min−1, and an IM gas flow of 90 mL min−1. The data were acquired in positive ion mode from 50 to 2000 Da with a 1 s scan time; leucine enkephalin was used as the lock mass (m/z 556.2771). All runs were analyzed using HDMSE with an energy ramp from 10 to 40 eV.

Metabolite Data Processing and Analysis

The acquired UPLC-IM-MSE data were imported, processed, normalized and interpreted in Progenesis QIv.2.1 (Non-linear Dynamics, Newcastle, UK). Briefly, each UPLC-IM-MSE data file was imported as an ion intensity map (used for visualization in both m/z and retention time dimensions) and underwent retention time alignment and peak picking. Peak picking was performed on individual aligned runs by matching peaks in an aggregate data set that is created from all aligned runs. Following peak picking, the features (retention time and m/z pairs) were reduced using both adduct ([M+H]+, [M+Na]+, [M+K]+, etc.) and isotope deconvolution. Data were normalized to all compounds. Statistically significant changes were identified using multivariate statistical analysis including principal component analysis (PCA) and p-values generated using analysis of variance (ANOVA) or pairwise comparisons. Pairwise comparisons were performed for each cisplatin treatment (1, 6, 24 or 48 h) vs. its matched control (1, 6, 24 or 48 h). Three biological and three technical replicates from each sample type were used to calculate both fold change and p-value and features were considered for identification only if they met both significance criteria of fold change ≧|1.5| and p≦0.1; this list was designated as ‘prioritized metabolites’. Prioritized metabolites or features were assigned tentative structural identifications using accurate mass measurements (<10 ppm error) and isotope distribution by searching the Human Metabolome Database (HMDB) (Wishart et al., Nucleic Acids Res. 41, D801-D807 (2013)). Following tentative structural identifications for both chromatography methods (HILIC and RPLC), spreadsheets were merged for further data processing. In particular, metabolites associated with drugs, plants, food, and microbial origin were eliminated. Metabolites with a tentative structural identification (met the dual significance criteria of fold change at an absolute value of 1.5 or above and a p-value of ≦0.1) were used in the mechanism of action. In an effort to increase the confidence in metabolite assignment, fragmentation spectra of metabolites that met significance criteria were searched in HMDB, METLIN (Smith et al., Ther. Drug Monit. 27, 747-751 (2005)), MassBank (Horai et al., J. Mass Spectrom. JMS 45, 703-714 (2010)), and NIST (The National Institute of Standards and Technology, ChemData. NIST. Gov. Mass Spectrometry Data Center 2014). Metabolite peak identifications were putatively assigned using product ions observed in the fragment ion spectra analyzed in HDMSE mode. Ion mobility separations were used to isolate precursor ions and correlate product ions.

Computational Analysis

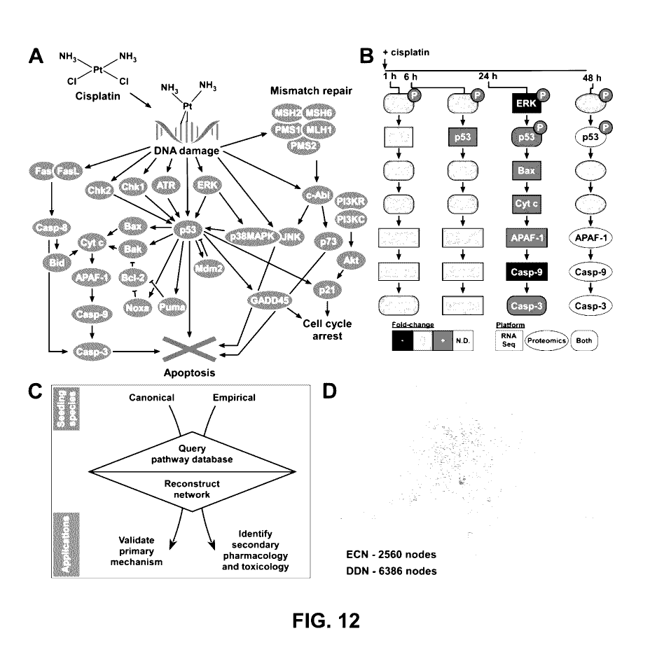

Data from all platforms were integrated and parsed for significantly changed, unique species for comparison against the canonical cisplatin mechanism and for network analysis. An analysis pipeline implemented in the Python programming language as part of the PySB modeling framework was developed. Bioservices was used to download pathways from the KEGG database that contain any proteins from a list of seed species. These pathways were then combined to form a unified network based on common protein species. Using this approach, they built three distinct networks. The expanded canonical network (ECN) was based on species in the cisplatin canonical mechanism (FIG. 12A). The expanded resistance network (ERN) was based on seed species involved in cisplatin resistance collated from a literature search. The data driven network (DDN) was based on significantly changed, unique species measured in the high-dimensional-omics data. Venn diagrams were made using eulerAPE (Micallef and Rodgers, PLOS ONE 9, e101717 (2014)).

Data Mining

To examine the species-to-species interactions in their data networks, the open source systems biology platform Cytoscape was used. The data network was uploaded and queried by selecting a species of interest and its viewing first-degree neighbors. Once connections were formed between species, pathways took form that were supported by using the QIAGEN IPA network analysis tool and annotated literature.

### Results

This study validates a multi-omics platform designed to assess the comprehensive MOA of exogenous compounds in 30 days. Selection of cell type, exposure methods, and analytical modalities were considered by evaluating stability, reproducibility, utility, and feasibility within 30 days. For this study, A549 cells were used; however, the platform is amenable to various adherent and suspension cell lines. The sponsoring agency selected cisplatin as the test compound and revealed its identity on the first day of the 30-day period.

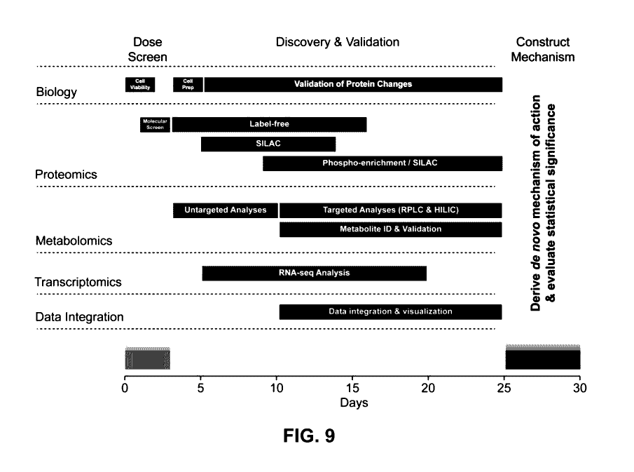

FIG. 9 graphically illustrates the three phases of the procedure: 1) molecular screening (days 0-3), 2) discovery analytics (days 4-25), and 3) mechanism construction (days 26-30). Phase 1 screens a wide range of cisplatin dose and exposure times to establish the treatment protocol for discovery experiments. This preliminary screen deduces exposure conditions that provide relevant data for the MOA, allowing the application of this protocol to uncharacterized compounds. During Phase 2, transcriptomics, proteomics, and metabolomics determine changes in molecular expression correlated with exposure to the compound. In Phase 3, data integration and analysis drive mechanism construction.

### Phase 1: Preliminary Screening Determines Relevant Dose and Exposure Time

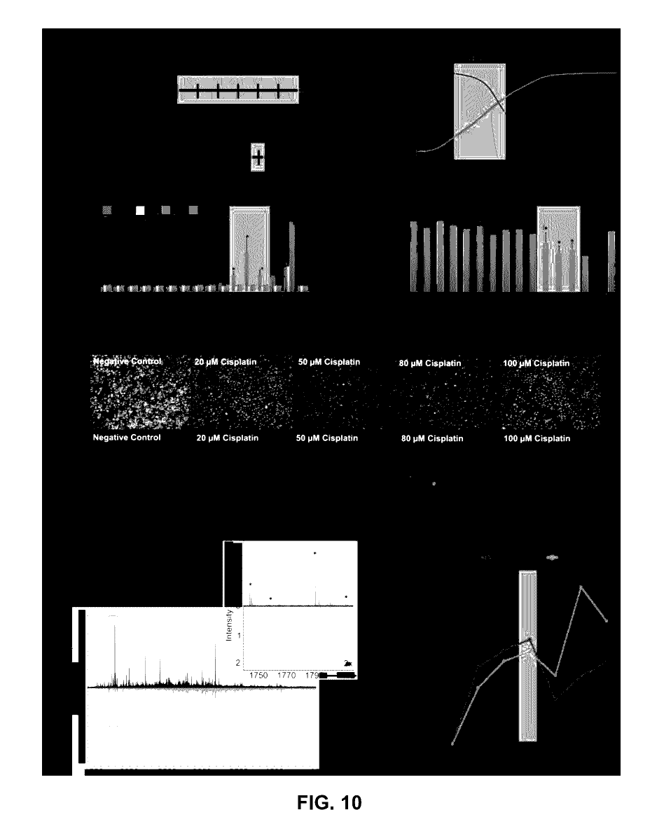

To make the analysis strategy applicable to uncharacterized compounds, it does not rely on previous experimental data to establish an exposure dose. It was hypothesized that a preliminary screening process (FIG. 10) could select optimal treatment conditions for Phase 2 experiments. FIG. 10A illustrates the two-stage protocol. First, a set of assays that indicate physiological perturbations (e.g., cell viability, etc.) narrows the possible dose range to a small number of conditions. Second, mass spectrometry (MS) analysis of proteome changes within this limited dose range leads to the selection of a single exposure condition for all subsequent experiments. The optimal dose, conceptualized in FIG. 10B, elicits maximum molecular response while preserving >50% cell viability.

During Stage 1 of the screening experiment, dose-dependent caspase 3/7 activation (FIG. 10C), ATP levels (FIG. 10D), and cell viability (FIG. 10E) were monitored. 14 doses (0.025-200 μM) were analyzed for cisplatin exposure times of 1, 6, and 24 h, with some selected measurements at 48 and 96 h. Based on the results obtained on day 1, the dose range of 20-100 μM was prepared for molecular screening.

For Stage 2, a rapid proteome screen using matrix-assisted laser desorption ionization (MALDI) MS was developed to evaluate the magnitude of the molecular response. This assay determines changes in MS profiles at selected conditions compared to control, ensuring maximum opportunity to observe significant molecular changes in the discovery phase. To maximize throughput, the inventors focused on profile changes rather than identifications, avoiding the use of chromatography and tandem MS. These results titrated the cisplatin dose used in later experiments but were not used in the construction of the cisplatin MOA.

FIG. 10F shows representative mass spectra from this experiment—a 6 h exposure of 50 μM cisplatin and a vehicle control. Each peptidic profile contained >4,000 unique mass-to-charge (m/z) peaks to monitor for intensity changes across exposure conditions. To determine which cisplatin dose induced the greatest molecular change, a quantitative, automated approach was developed to determine a molecular variance score—a metric that projects the high-dimensional variance between the spectra of different experimental conditions to a lower-dimensional representation using a principle component analysis.

FIG. 10G shows the molecular variance score for each cisplatin dose measured at 1 and 6 h. Only exposure times <24 h were analyzed during the molecular screen to maintain an efficient screening period (≦3 days). An increase in molecular variance was observed with increasing cisplatin dose up to 50 μM; greater doses did not show a correlated response. This instability in the molecular variance score at doses >50 μM at 1 and 6 h corresponds with the variation seen in the caspase activation assay in the same dose range at 24 h (FIG. 10C). In both the physiological and molecular screen, 50 μM cisplatin elicits a maximum response and maintains cell viability of >50%, indicating an optimal dose of 50 μM cisplatin for discovery experiments.

This screen-determined concentration compares with reports of cisplatin induced apoptosis and cytotoxicity; exposure doses range from 3.3-1000 μM, and the IC50 for A549 cells is 18-64 μM. Yang et al., PloS One 8, e65309 (2013). The molecular screen confirmed cisplatin-induced toxicity is measurable at 50 μM and as early as 1 h. These results support our hypothesis that a preliminary screen can determine optimal treatment conditions for an unknown compound, and they validate our established workflow.

### Phase 2: A Multi-Omics Platform Captures Cisplatin-Induced Molecular Perturbations

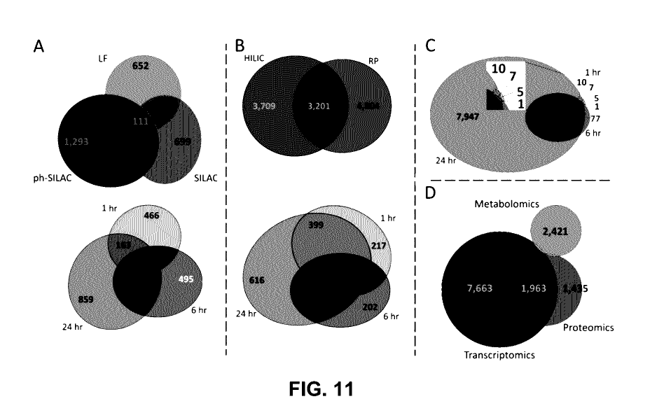

During the discovery phase, comprehensive molecular data was acquired from transcriptome, proteome, phosphoproteome, and metabolome measurements of A549 cells treated with 50 μM cisplatin for 1, 6, 24 and 48 h. In total, 254,470 measurements were collected. Of the 53,500 unique, individual species detected, 13,483 were significantly changed (24%). FIG. 11 shows the data generated by these modalities. Integration of data from these platforms facilitated the de novo, time-resolved MOA construction described below.

### Phase 3: Mechanism Construction

Comparison of the Empirical Data to a Canonical Cisplatin Mechanism

To evaluate the dataset, a canonical MOA was generated consisting of 33 species from a literature survey of transcripts, proteins, and metabolites that change in a variety of cell lines exposed to cisplatin for less than 48 h. FIG. 12A shows the constructed canonical mechanism; cisplatin-induced DNA damage initiates a cellular response that ends in apoptosis. The multi-omics platform detected 97% of the species in the canonical cisplatin MOA (all excepting Mdm2), and 82% of these changed significantly.

FIG. 12B illustrates time-resolved data for an intrinsic apoptosis pathway within the canonical mechanism. Phosphorylation at Thr202 and Tyr204 activates ERK1; the homologous motif on ERK2 is Thr185/Tyr187. Phosphorylated ERK1 and/or ERK2 increased at each time point. Activated ERK can phosphorylate and activate p53 at multiple sites, including Ser392, which increased significantly in cisplatin-treated cells beginning at 24 h. The literature shows that p53 binds DNA as a tetramer, and phosphorylation at Ser392 enhances tetramer formation 10-fold. Additionally, Chk1 or Chk2 can phosphorylate p53 at Ser313/Ser314. The data show that p53 pSer313, pSer314 and/or pSer315 increased significantly in cisplatin-treated cells starting at 6 h. Phosphorylation at these sites can activate BAX, consistent with increased detection of the BAX transcript in cisplatin-treated cells at 24 h. In intrinsic apoptosis, Bax conformational change in the mitochondrial membrane contributes to cytoplasmic release of Cyt 210 c (CYCS) leading to assembly of the apoptosome, which includes APAF-1 and Casp-9, and subsequent Casp-3 activation. Transcriptional upregulation was observed in BAX, CYCS, APAF1, and CASP3 but not proteomic abundance changes for these species, consistent with mediation of their MOA through conformational changes, localizations, and cleavage events. Due to the central role of Casp-9 in the caspase cascade, downregulation of CASP9 at 24 h is consistent with a sub-population of cells initiating anti-apoptotic pathways. By 48 h, none of the downstream apoptosis proteins changed significantly, suggesting that surviving cells were not initiating apoptosis.

Comparison of the Empirical Data to Expanded Mechanisms

Comparison of the measured data to the literature-derived canonical MOA demonstrates agreement both in the network and on a time-resolved basis. However, the capture of 32 out of 33 species from >53,000 unique measurements tests less than 0.1% of the collected data. Thus, further validation of our approach required a strategy that expands beyond the current literature.

Networks were constructed by seeding with inputs based on annotated biology, with expansion informed by curated pathways from the Kyoto Encyclopedia of Gene and Genomes (KEGG) (Kanehisa et al., Nucleic Acids Res. 40, D109-114 (2012)), allowing them to validate their empirical findings against expected outcomes. The comprehensive nature of multi-omics datasets can surpass previously described MOAs. Therefore, the inventors hypothesized that seeding the empirically captured dataset would allow us to move beyond these limits and permit exploration of previously unknown but important cellular and pharmacological events associated with the exposure conditions. FIG. 12C illustrates this concept. Two networks were developed to validate and interrogate the empirical dataset: the expanded canonical network (ECN), seeded with species from the canonical mechanism, and the data driven network (DDN), seeded with unique significantly changed species from our empirical data, 11,061 species.

The ECN contained 2,560 unique species (FIG. 12D). The multi-omics dataset captured 1,397 of these (55%), of which 1,229 changed significantly. The percentage of unique species that changed significantly in the ECN, 88%, is approximately 3.5 fold higher than the percentage of significantly changed species in the empirical dataset. This value is also similar to the percent of significantly changed species determined for the canonical mechanism, validating the primary MOA on the scale of thousands of species and revealing the relevance of the ECN to the cisplatin MOA. Still, the significantly changed multi-omics data contains 80% more species than the ECN, providing the opportunity to investigate unexplored pathways related to cisplatin treatment. The inventors hypothesized that these additional measurements revealed previously described and novel off-target effects of cisplatin, including resistance mechanisms.

The resulting DDN had 6,386 species. The inventors experimentally measured 2,583 (40%) of these species, 2,215 of which were significantly changed. Similar to the ECN, 86% of the detected species within the DDN changed significantly. For the DDN, limitations to KEGG precluded seeding with metabolomics data and limited the transcriptomic and proteomic data to the 2,215 species that were in KEGG (F. FIG. 12D shows the ECN and DDN, with a total of 2,560 species (nodes) overlapping. The uncovered DDN region represents intracellular responses not currently understood in the context of cisplatin. As hypothesized, the empirical dataset provides the opportunity to explore novel secondary mechanisms.

### Beyond the Primary MOA

Capturing the Dynamic Processes that Govern Cell Fate

To complement the DDN analysis and investigate the capability of the platform to capture events outside of the primary MOA, the inventors threaded the empirical data through the Qiagen IPA causal network analysis tool and analyzed for pathways present at every time point. They selected the HUWE1 pathway (Zhong et al., Cell 121, 1085-1095 (2005)) for further analysis since it appeared as one of the top five ranked hits at every time point and was the top hit at 6 h. The HUWE1 network contained a number of empirically measured species that revealed significant fold changes over time.

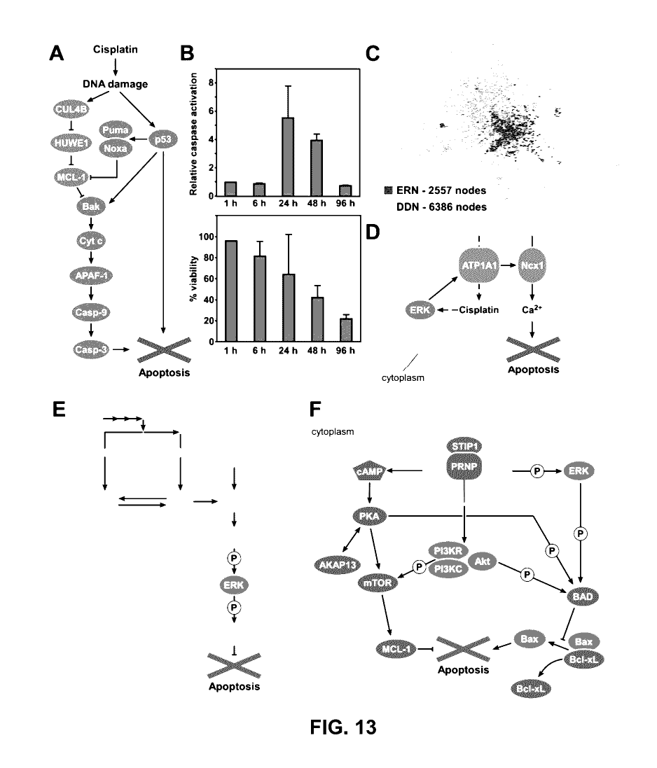

HUWE1 is an E3 ubiquitin ligase that modulates DNA damage response and apoptosis pathways upon genotoxic stress. HUWE1 targets MCL-1, an anti-apoptotic Bcl2 family member, for ubiquitination and proteasomal degradation; this alleviates MCL-1 repression of Bak and allows Bak to drive pore formation in the mitochondrial outer membrane (FIG. 13A). Pore formation induces the release of Cyt c into the cytosol and initiates the caspase cascade. A recent publication shows that upregulation of CUL4B leads to increased degradation of HUWE1 and subsequent stabilization of MCL-1, which inhibits Bak by forming a heterodimer and ultimately steers the cell away from apoptosis.

The regulation of this pathway was captured in a time-resolved manner. The CUL4B C-terminal peptide increased at 1 h with a concomitant significant change in HUWE1 phosphorylation. At 6 h post-exposure CUL4B was unchanged, but we observed evidence for a decrease in HUWE1 phosphorylated and unphosphorylated states. Analysis at 24 and 48 h revealed multiple significant abundance changes for phosphorylated peptides of HUWE1 in treated cells, indicating a dynamic regulation process. Additionally, HUWE1 decreased in abundance at 48 h, suggesting that a population of cells were resistant. Although we did not observe MCL-1 at any time point, BAK1 increased at 24 h suggesting that a population of cells were committed to apoptosis. The temporal nature of this pathway highlights the dynamic processes at play in the cisplatin-exposed population, with an apparent early upregulation of pro-survival mechanisms, a later commitment to apoptosis, and detection of an emerging resistant population at 48 h.

Given the dynamic nature of the HUWE1 regulatory circuit, apoptosis and viability pathways were explored using IPA and these findings were compared to the empirically derived kinetics of caspase activation and viability. At each time point, IPA sorted significantly changed species in the dataset into apoptosis-inhibiting or—activating categories based on their upregulation or downregulation and correlation with known functions. The total number of apoptotic proteins increased up to 24 h and declined by 48 h. However, the ratio of activating to inhibiting molecules remained stable at each time point (52-54%), revealing that not all molecular changes are pro-apoptotic. Similar results were observed when IPA sorted species into viability-inhibiting or -activating categories. The total number of proteins in the viability pathway increased up to 24 h and declined by 48 h, but the ratio of anti- to pro-survival species remained constant (52-57%). These trends suggest a heterogeneous population of cells engaged in the dynamic processes of committing to apoptosis or survival.

Consistent with IPA analysis, the measured cellular responses revealed maximal caspase activation at 24 h (FIG. 13B). Both IPA analysis and measured cellular data indicate that caspase activation ceased after 48 h. Indeed, the viability data in FIG. 13B show a small population of cells (c., 20%) persisted to 96 h, suggesting that these cells represent a cisplatin-resistant population. This prompted the inventors to determine if the platform had captured known and novel resistance mechanisms. Such a capability would provide an early indication of drug resistance mechanisms and/or off-target effects—critical knowledge that could improve clinical trial outcomes.

### Construction of Cisplatin Resistance Mechanisms

To validate the capture of resistance mechanisms, the inventors seeded an expanded resistance network (ERN) using the same approach as for the ECN. They identified six proteins in KEGG known to play a role in cisplatin resistance. Their expansion resulted in the 1,236-species ERN. The empirical data contained 667 (54%) of these, 589 of which changed significantly. Thus, 88% of the empirically detected species in the ERN changed 325 significantly. FIG. 13C shows the ERN overlaid on the DDN. The significant overlap of matching nodes validates the presence of known resistance pathways in the dataset, consistent with a putatively resistant population at 96 h. Thus, the ERN was utilized to generate testable molecular hypotheses for previously reported resistance proteins.

### ATP1A1 Mediated Resistance

ATP1A1, a seed for the ERN and a significantly changed species within the inventors' empirical dataset, regulates cisplatin uptake into cells and modulates resistance when its expression is suppressed (O'Grady et al., Cancer Treat. Rev. 40, 1161-1170 (2014)). Interestingly, ATP1A1 also regulates activity of Ncx1. Abrogation of ATP1A1 function concomitantly attenuates Ncx1 activity, which perturbs the calcium signaling pathways of the cell, a phenomenon associated with evasion of apoptosis and implicated in cancer. FIG. 13D shows the ATP1A1 and Ncx1 pathway. ATP1A1 peptides decreased significantly at 6 and 24 h, consistent with enhanced resistance over time. Ncx1 antisense RNA increased at 1 h, with a significant decrease in the Ncx1 transcript measured at 6 h. At 24 h, both the Ncx1 antisense RNA and the Ncx1 transcript decreased. Reduction of Ncx1 expression and function at these early time points may provide escape avenues by perturbing downstream calcium signaling pathways and disrupting the apoptotic circuit. Recently, disruption of intracellular calcium signaling, tolerance of ER stress, and reduced expression of a subunit of calcium-regulated big potassium channels were implicated in cisplatin resistance. Samuel et al., Tumour Biol. 37, 2565-2573 (2016). Therefore, a better understanding of the pathways that disrupt calcium homeostasis and calcium-regulated apoptotic events is critical to further elucidate cisplatin resistance. Data derived from our multi-omics dataset for ATP1A1 and Ncx1 present a potential mechanism.

### Estrogen-Induced Cisplatin Resistance

Recently, estrogen was shown to mediate resistance to cisplatin-induced apoptosis in A549 cells. Grott et al., Anticancer Res. 33, 791-800 (2013). While this study highlighted the importance of caspase attenuation in the mechanism, it did not elucidate a detailed molecular process. The estrogen hormones estrone (E1) and estradiol (E2) are synthesized from androgens by aromatase and can also be interconverted by HSD17βs. Additionally, estrogen receptors ESR1 and ESR2 cooperate in promoting early activation of ERK. Examination of the estrogen-related pathways in the inventors' dataset revealed a transient metabolomic response to cisplatin-induced cyototoxic stress that ultimately leads to a protein-based resistance mechanism. FIG. 13E demonstrates a network of events derived de novo from measured molecular changes that potentially lead to resistance through mTOR activation. In cisplatin-treated cells, estrogen species transiently increased: E1 increased at 1 h, and both E1 and E2 increased at 6 h but decreased by 24 h. HSD17β7 increased at 1 h and decreased at 6 h. The transcription factor C/EBPβ, which is activated by cyclic AMP (cAMP)-dependent protein kinase A (PKA), regulates HSD17β dehydrogenase family members. At 24 h, an increase in PKA and in HSD17β7 transcript was observed. E2 binds to ESR1 and 371 induces PKC-mediated ERK phosphorylation and ERK-dependent mTOR activation. The inventors observed ERK phosphorylation at every time point, with mTOR phosphorylation increased at 24 h.

FIG. 13E also shows the interaction of STAT1 with ESR leading to mTOR activation. STAT1 overexpression mediates cisplatin resistance in ovarian cancer cell lines through an as yet unexplained mechanism. Roberts et al., Br. J. Cancer, 92, 1149-1158 (2005). Activated STAT1 induces the expression of ESR1, feeding into the above described PKC-ERK mediated activation of mTOR and leading to resistance. Taken together, these data suggest that the STAT1 and estrogen-mediated cisplatin resistance pathways are complementary and that the key elements of the estrogen signaling pathway are activated by 24 h, which may allow escape from cisplatin-induced cytotoxicity by a unique ERK/mTOR axis.

### Mining Novel Mechanisms of Cisplatin Resistance

While the ERN guided identification of associated resistance molecules, pathways outside of the ERN provide the opportunity to discover resistance mechanisms de novo. To explore this, the inventors analyzed the top 20 most dynamically regulated proteins at each time point for potential contribution to mechanisms of resistance and sorted the data based on known links to proliferative capacity or apoptosis.

The STIP1 Cascade

FIG. 13F illustrates a network of events culminating in potential apoptotic escape mechanisms derived de novo from analysis of measured events in the multi-omics dataset. Based on a dynamic change at 1 h, the inventors identified stress inducible protein 1 (STIP1) as a putative resistance marker. The DDN associates STIP1 with the prion protein PRNP, which links to the apoptotic activator Bax. Additionally, STIP1 and PRNP associate with cAMP, and activation of the ERK1/2 pathway requires PRNP and STIP1 endocytosis. STIP1 binds to PRNP to drive cell proliferation via activation of the MEK/ERK and PI3K pathways. Collectively, this implicates the PI3K, ERK1/2, and cAMP transduction pathways as downstream modulators of the STIP1-PRNP interaction.

Unification of the synergistic activities of PI3K, ERK1/2, and PKA culminating in BAD phosphorylation presents a novel mechanism elucidated de novo from our empirical data. The right side of FIG. 13F illustrates the ERK-mediated signaling events. As previously discussed, increased levels of activated ERK 1/2 were detected at every time point and likely contribute to apoptosis. However, activated ERK can also contribute to anti-apoptotic pathways via phosphorylation of BAD, emphasizing its pleiotropic effects. The left side of FIG. 13F shows PKA-mediated signaling events. Transcription of catalytic subunits of PKA changed dynamically, with PRKACA upregulated and PRKACB downregulated at 24 h. Functionally, PKA is anchored by binding the AKAP family of proteins, and in the inventors' dataset AKAP13 levels as well as phosphorylation states were dynamically regulated at every time point with overall levels up significantly by 48 h. The center of FIG. 13F displays PI3K mediated signaling events.

Within this pathway, a number of significant changes were detected at 24-48 h consistent with proliferation in a population of cells. The downstream targets of these pathways, mTOR and BAD, also changed significantly at later time points. Phosphorylated mTOR at pS1166 increased at 24 h. This phosphorylation event was previously identified in response to the pro-proliferative IGF stimulus, consistent with a role in anti-apoptotic signaling. Additionally, increased BAD phosphorylation was detected at residues that prevent its binding to Bcl-xL/Bcl-2: pSer75 (mediated by ERK1/2) at 48 h and pSer118 (mediated by PKA) at 24 h and at 48 h.

Further analysis of the species in these pathways provides insight into cancer development and drug resistance. Overexpression of PRNP in colorectal cancer cells enhances proliferation and attenuates doxorubicin-induced apoptosis. Additionally, PRNP upregulates the transcriptional activity of β-catenin/TCF4, which inhibits apoptosis upon cisplatin exposure. Increased levels of cAMP also confer protection against cisplatin-induced DNA damage and apoptosis, likely through PKA activity. STIP1 is a biomarker for many carcinomas, and it is most commonly associated with ovarian cancers. Cell surface interaction of STIP1 and PRNP was first identified as a neuroprotective event that rescued neurons from apoptosis. Subsequently, it was determined that neuroprotection is mediated by increasing protein synthesis via the PI3K/mTOR signaling axis.

In summary, PI3K, ERK1/2, and cAMP via PKA converge on pro-apoptotic BAD and modulate its phosphorylation. Phosphorylated BAD does not bind and displace Bcl-xL or Bcl-2 from Bak/Bax, preventing Bak/Bax-mediated apoptosis. PI3K and PKA also stimulate mTOR, resulting in stabilization of MCL-1 and further inhibition of the apoptotic pathway by sequestration of Bak. As a whole, the STIP1 cascade presented in FIG. 13F ultimately targets mTOR and BAD, disrupting both Bak and Bax and protecting the cell from apoptosis through inhibition of pore formation in the mitochondrial outer membrane.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method comprising the steps of:
exposing one or more animal cells to a chemical agent;
generating data representing an altered molecular phenotype of the one or more animal cells after exposure to the chemical agent using a multi-omics analysis;
providing the data representing the altered molecular phenotype to a system comprising a processor;
using the system to compare the data representing the altered molecular phenotype to data representing a normal molecular phenotype of the one or more animal cells; and
using the system to output a characterization of a cellular response of the one or more animal cells to the chemical agent based on results of the comparing the data representing the altered molecular phenotype of the one or more animal cells to data representing the normal molecular phenotype of the one or more animal cells.

2. The method of claim 1, wherein the cellular response is characterized in 30 days or less of exposing the one or more animal cells to the chemical agent.

3. The method of claim 1, wherein the multi-omics analysis comprises desorption ionization mass spectrometry analysis.

4. The method of claim 3, wherein the desorption ionization mass spectrometry comprises matrix-assisted laser desorption/ionization (MALDI) mass spectrometry.

5. The method of claim 3, wherein the desorption ionization mass spectrometry comprises electrospray ionization (ESI) spectrometry.

6. The method of claim 1, wherein the multi-omics analysis comprises at least one of proteomic analysis, metabolomic analysis, and transcriptomic analysis.

7. The method of claim 1, wherein the data representing the normal molecular phenotype of the one or more animal cells is obtained concurrent with the data representing the altered molecular phenotype using corresponding isotopically labeled animal cells.

8. The method of claim 1, further comprising the step of screening the one or more animal cells to select optimal treatment conditions before generating the data representing the altered molecular phenotype of the one or more animal cells after exposure to the chemical agent using the multi-omics analysis.

9. The method of claim 1, wherein the one or more animal cells have been cultured in a well plate having a plurality of wells.

10. The method of claim 9, wherein the one or more animal cells in the well plates are analyzed using high-throughput screening.

11. The method of claim 1, wherein the one or more animal cells comprise an animal tissue sample.

12. The method of claim 1, wherein the chemical agent is at least one of a pharmaceutical agent, a threat agent, a biologic agent, and a toxic agent.

13. The method of claim 1, wherein a plurality of animal cells are exposed to different concentrations of the chemical agent.

14. The method of claim 1, wherein the normal molecular phenotype of the one or more animal cells is limited to a molecular phenotype associated with a specific biological pathway so that the chemical analysis indicates at least one previously unknown mechanism.

15. The method of claim 1, wherein the chemical agent comprises an unknown toxic agent.

16. A system comprising:
a non-transitory memory storing computer-executable instructions; and
a processor to execute the computer-executable instructions to at least:
receive data generated by a multi-omics analysis representing an altered molecular phenotype of one or more animal cells after exposure to a chemical agent;
compare the data representing the altered molecular phenotype with data representing the normal molecular phenotype of the one or more animal cells; and
output, to a display device, a characterization of the cellular response of the one or more animal cells to the chemical agent based on results of comparing the data.

17. The system of claim 16, further comprising an apparatus for exposing one or more animal cells to the chemical agent.

18. The system of claim 17, wherein the apparatus comprises a well plate having a plurality of wells.

19. The system of claim 18, wherein the apparatus further comprises a high throughput apparatus including a robot arm for analysis of the plurality of wells.

20. The system of claim 16, wherein the multi-omics analysis comprises desorption ionization mass spectrometry analysis.

21. The system of claim 20, wherein the desorption ionization mass spectrometry comprises matrix-assisted laser desorption/ionization (MALDI) mass spectrometry.

22. The system of claim 20, wherein the desorption ionization mass spectrometry comprises electrospray ionization (ESI) spectrometry.

23. The system of claim 16, wherein the multi-omic analysis comprises at least one of proteomic analysis, metabolomic analysis, and transcriptomic analysis.

24. The system of claim 16, wherein the display device provides a visualization of the characterization.
